SEQUENCE LISTING (32 sequences)

SEQ ID NO: 1 - DNA, 51 bp, Homo sapiens
ttggtgtaaacacctgggaactctgvttttccacagttttccccccaactc

SEQ ID NO: 2 - DNA, 51 bp, Homo sapiens
tacatgcatcgtttctggctggggaygagaaaaaggtatatgttttacagt

SEQ ID NO: 3 - DNA, 51 bp, Homo sapiens
ggaacgaggcagctcacaatcttttytgtttccgtcttttttcatttcaat

SEQ ID NO: 4 - DNA, 51 bp, Homo sapiens
gaatacgtggatagaattattttccwtgaaaactacaatgcaggcacttac

SEQ ID NO: 5 - DNA, 51 bp, Homo sapiens
cctgcattgtagttttcatggaaaakaattctatccacgtattcaattact

SEQ ID NO: 6 - DNA, 51 bp, Homo sapiens
tctatccacgtattcaattactataygtttaaggtcggggtgtatccagtc

SEQ ID NO: 7 - DNA, 51 bp, Homo sapiens
tattcaattactatacgtttaaggtyggggtgtatccagtctactactgtt

SEQ ID NO: 8 - DNA, 51 bp, Homo sapiens
ctcttcctttcttgaagaaagaagayggataaaatcattattacctaaact

SEQ ID NO: 9 - DNA, 51 bp, Homo sapiens
attttaataaaaattttaccttgagycacagatgcaaagcctgaagaaaac

SEQ ID NO: 10 - DNA, 51 bp, Homo sapiens
acccgatttgcaatggaagcctttgbcttggcatgctgtgcaaacataagc

SEQ ID NO: 11 - DNA, 51 bp, Homo sapiens
tccacaatcattgataccatcacagsctttcatctgagaaatgtatttccc

SEQ ID NO: 12 - DNA, 51 bp, Homo sapiens
atcagcgaaatcctggtaacccatarttcttctcttagtaaaagtacattc

SEQ ID NO: 13 - DNA, 51 bp, Homo sapiens
agcgaaatcctggtaacccatagttyttctcttagtaaaagtacattcagc

SEQ ID NO: 14 - DNA, 51 bp, Homo sapiens
ttcagccaaactggtctctaatcctyggcaatgcacatgtagacattcagt

SEQ ID NO: 15 - DNA, 51 bp, Homo sapiens
actattccctctgaatctgtatttcyatgcttcaaggaaacactaaacttt

SEQ ID NO: 16 - DNA, 51 bp, Homo sapiens
gtgccctcaatgcatctctgccatgsctggcagaagactttatcgcaggag

SEQ ID NO: 17 - DNA, 52 bp, Homo sapiens
gaccgtccggcccacgggtagcagctkgtggttgacggtgaagatccgatag

SEQ ID NO: 18 - DNA, 52 bp, Homo sapiens
ctcattgtcaaaaggtgtgcttagggratccatccctaaaatgttgatcctg

SEQ ID NO: 19 - DNA, 22 bp, Artificial Sequence
  Description of Artificial Sequence Synthetic oligonucleotide
gataaaacaaacaatataacag

SEQ ID NO: 20 - DNA, 22 bp, Artificial Sequence
  Description of Artificial Sequence Synthetic oligonucleotide
aataaaacaaacaatataacag

SEQ ID NO: 21 - DNA, 22 bp, Artificial Sequence
  Description of Artificial Sequence Synthetic oligonucleotide
ggtaaaataaacagcgttacag

SEQ ID NO: 22 - DNA, 22 bp, Artificial Sequence
  Description of Artificial Sequence Synthetic oligonucleotide
ggccggacaaacaatataacag

SEQ ID NO: 23 - DNA, 22 bp, Artificial Sequence
  Description of Artificial Sequence Synthetic oligonucleotide
ggccggctggtaagcgctacag

SEQ ID NO: 24 - DNA, 22 bp, Artificial Sequence
  Description of Artificial Sequence Synthetic oligonucleotide
agccggctggtaagcgctacaa

SEQ ID NO: 25 - DNA, 22 bp, Artificial Sequence
  Description of Artificial Sequence Synthetic oligonucleotide
ggccggctgatacgcgttatag

SEQ ID NO: 26 - DNA, 22 bp, Artificial Sequence
  Description of Artificial Sequence Synthetic oligonucleotide
ggccggctgatacgcgttacag

SEQ ID NO: 27 - DNA, 22 bp, Artificial Sequence
  Description of Artificial Sequence Synthetic oligonucleotide
ggtcggctgatacgcgttacgg

SEQ ID NO: 28 - DNA, 22 bp, Artificial Sequence
  Description of Artificial Sequence Synthetic oligonucleotide
ggtcggctgatacgcgttacag

SEQ ID NO: 29 - DNA, 22 bp, Artificial Sequence
  Description of Artificial Sequence Synthetic oligonucleotide
ggtcggctgatacgcgtttcag

SEQ ID NO: 30 - DNA, 22 bp, Artificial Sequence
  Description of Artificial Sequence Synthetic oligonucleotide
agtcggctgatacgcgttacag

SEQ ID NO: 31 - DNA, 22 bp, Artificial Sequence
  Description of Artificial Sequence Synthetic oligonucleotide
ggtcggctggtacgtgtaacag

SEQ ID NO: 32 - DNA, 22 bp, Artificial Sequence
  Description of Artificial Sequence Synthetic oligonucleotide
agtcggctggtacgcgtaacag

(19) United States
(12) Patent Application Publication
(10) Pub. No.: US 2014/0342919 A1
(43) Pub. Date: Nov. 20, 2014

(54) MARKERS RELATED TO AGE-RELATED MACULAR DEGENERATION AND USES THEREFOR

(71) Applicant: Johanna M. Seddon, Boston, MA (US)

(72) Inventor: Johanna M. Seddon, Boston, MA (US)

(21) Appl. No.: 14/203,073

(22) Filed: Mar. 10, 2014

Related U.S. Application Data
(60) Provisional application No. 61/775,673, filed on Mar. 10, 2013.
(60) Provisional application No. 61/778,601, filed on Mar. 13, 2013.

(51) Int. Cl. C12Q 1/68

(52) U.S. Cl. CPC C12Q 1/6883; C12Q 2600/156
USPC 506/2; 435/6.11; 506/9

(57) ABSTRACT

Methods are provided for determining a risk of age-related macular degeneration (AMD), including a risk of a subject developing AMD or a risk of a subject progressing to an advanced form of AMD based on the detection of rare variants in C3, C9, and CFI.

### RELATED APPLICATIONS

This application claims the benefit of U.S. Provisional Patent Applications Ser. Nos. 61/775,673, entitled Genes Associated with Progression to Advanced Stages of Macular Degeneration, filed on Mar. 10, 2013, and 61/778,601, entitled Markers Related to Age-Related Macular Degeneration and Uses Therefor, filed on Mar. 13, 2013, the contents both of which are incorporated herein by reference in their entireties for all purposes.

### STATEMENT REGARDING FEDERALLY FUNDED RESEARCH

This invention was made with government support under grant number ROI EY1 1309 awarded by the National Institutes of Health and the National Eye Institute. The government has certain rights in the invention.

### BACKGROUND

Age-related macular degeneration (AMD) is the most common geriatric eye disorder leading to blindness. Macular degeneration is responsible for visual handicap in what is estimated conservatively to be approximately 16 million individuals worldwide. Among the elderly, the overall prevalence is estimated between 5.7% and 30% depending on the definition of early AMD, and its differentiation from features of normal aging, a distinction that remains poorly understood.

Histopathologically, the hallmark of early neovascular AMD is accumulation of extracellular drusen and basal laminar deposit (abnormal material located between the plasma membrane and basal lamina of the retinal pigment epithelium) and basal linear deposit (material located between the basal lamina of the retinal pigment epithelium and the inner collageneous zone of Bruch's membrane). The end stage of AMD is characterized by a complete degeneration of the neurosensory retina and of the underlying retinal pigment epithelium in the macular area. Advanced stages of AMD can be subdivided into geographic atrophy and exudative AMD. Geographic atrophy is characterized by progressive atrophy of the retinal pigment epithelium. In exudative AMD the key phenomenon is the occurrence of choroidal neovascularisation (CNV). Eyes with CNV have varying degrees of reduced visual acuity, depending on location, size, type and age of the neovascular lesion. The development of choroidal neovascular membranes can be considered a late complication in the natural course of the disease possibly due to tissue disruption (Bruch's membrane) and decompensation of the underlying longstanding processes of AMD.

Many pathophysiological aspects as well as vascular and environmental risk factors are associated with a progression of the disease. Family, twin, segregation, and case-control studies all suggested an involvement of genetic factors in the etiology of AMD prior to the discovery of various genes associated with AMD.

Knowledge is growing about the extent of heritability, number of genes involved, and mechanisms underlying phenotypic heterogeneity. The search for genes and markers related to AMD faces challenges—onset is late in life, and there is usually only one generation available for studies. The parents of patients are often deceased, and their children are too young to manifest the disease. Generally, the heredity of late-onset diseases has been difficult to estimate because of the uncertainties of the diagnosis in previous generations and the inability to diagnose AMD among the children of an affected individual. Even in the absence of the ambiguities in the diagnosis of AMD in previous generations, the late onset of the condition itself, natural death rates, and small family sizes result in underestimation of genetic forms of AMD, and in overestimation of rates of sporadic disease. Moreover, the phenotypic variability is considerable, and it is conceivable that the currently used diagnostic entity of AMD in fact represents a spectrum of underlying conditions with various genetic and environmental factors involved.

There remains a strong need for improved methods of diagnosing or prognosticating AMD or a susceptibility to AMD in subjects, as well as for evaluating and developing new methods of treatment.

### SUMMARY

In an aspect of the present invention, a method is provided for determining a risk of age-related macular degeneration (AMD) in a human patient, the method including detecting in a sample from the human patient the presence of polymorphism rs147859257, and correlating the presence of polymorphism rs147859257 to an increased risk of AMD in the human patient.

In another aspect of the invention, a method is provided for determining a risk of age-related macular degeneration (AMD) in a human patient, the method including detecting in a sample from the human patient the presence of polymorphism rs34882957, and correlating the presence of polymorphism rs34882957 to an increased risk of AMD in the human patient.

In still another aspect of the invention, a method is provided for determining a risk of age-related macular degeneration (AMD) in a human patient, the method including detecting in a sample from the human patient the presence of a polymorphism listed in Supplementary Table 2, and correlating the presence of the polymorphism to AMD risk in the human patient.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

The present invention is illustrated by way of example and not limitation in the figures of the accompanying drawings, in which like references indicate similar elements and in which:

FIG. 1 is a graphical representation of aspects of the present invention;

FIG. 2 is a graphical representation of aspects of the present invention;

FIG. 3 is a graphical representation of aspects of the present invention;

FIG. 4 is a graphical representation of aspects of the present invention;

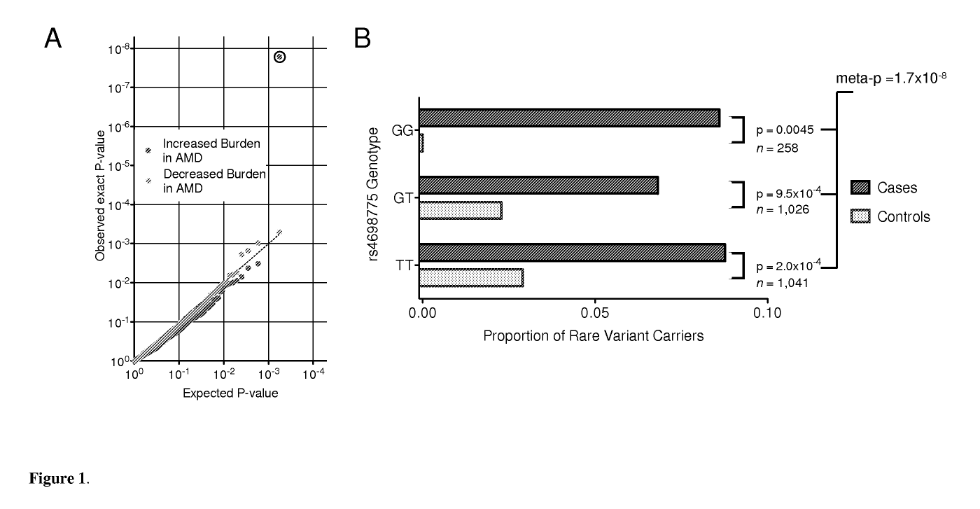

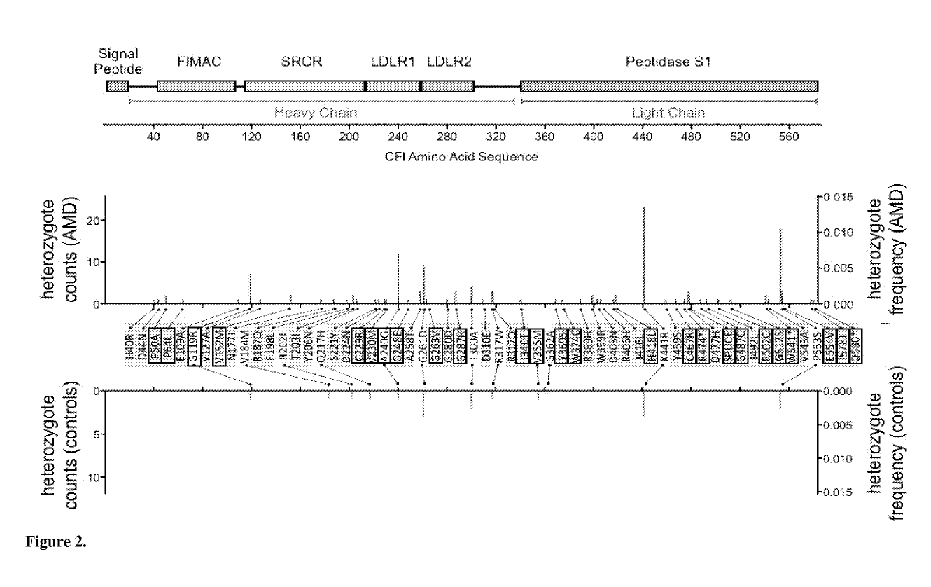

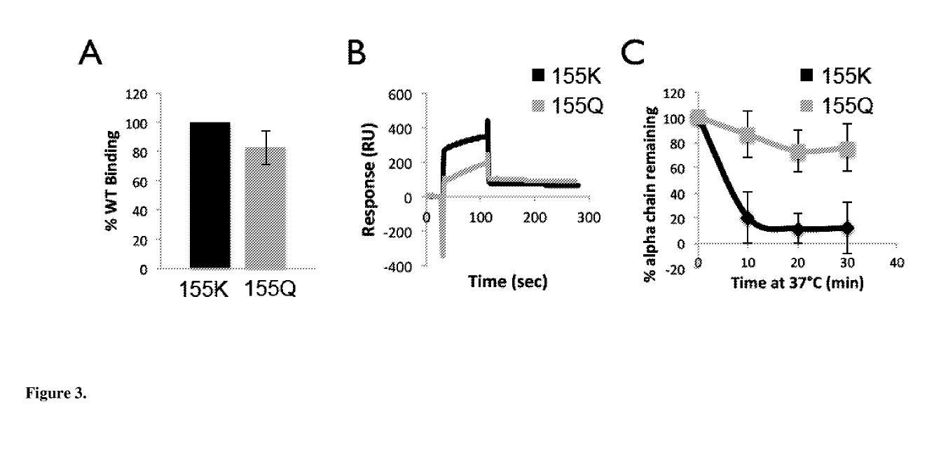

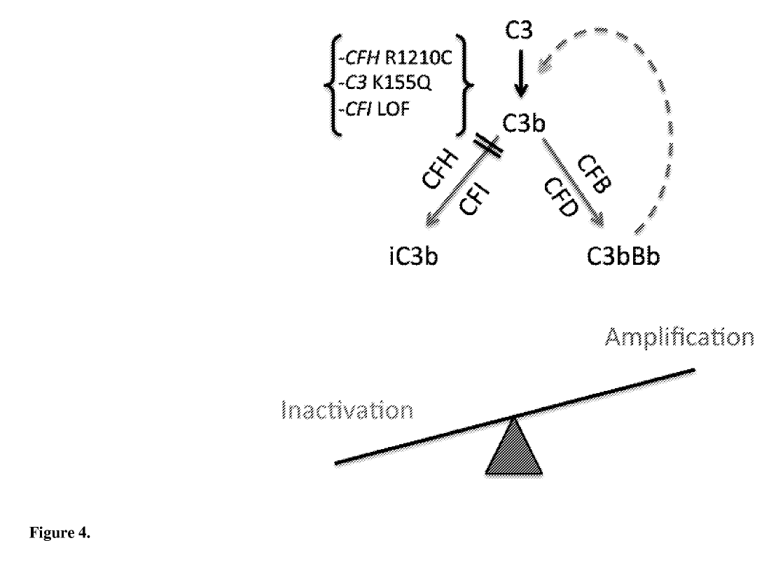

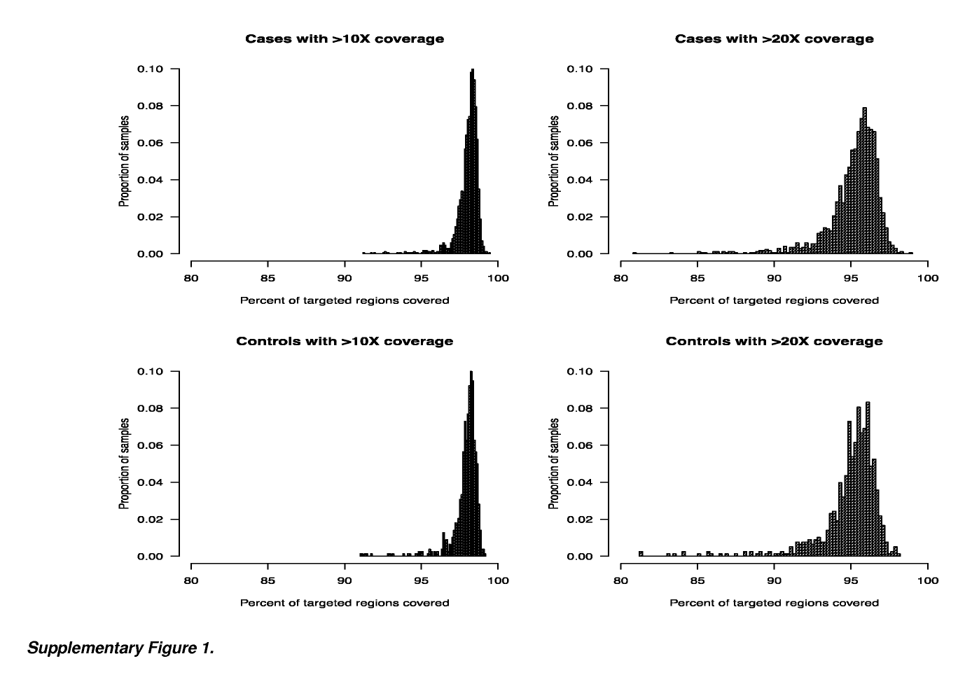

Supplementary FIG. 1 is a graphical representation of aspects of the present invention;

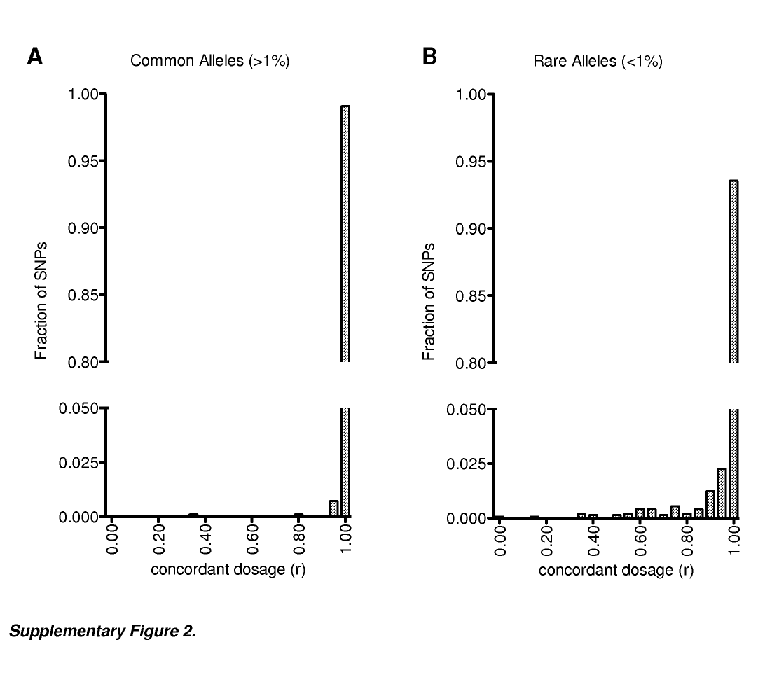

Supplementary FIG. 2 is a graphical representation of aspects of the present invention;

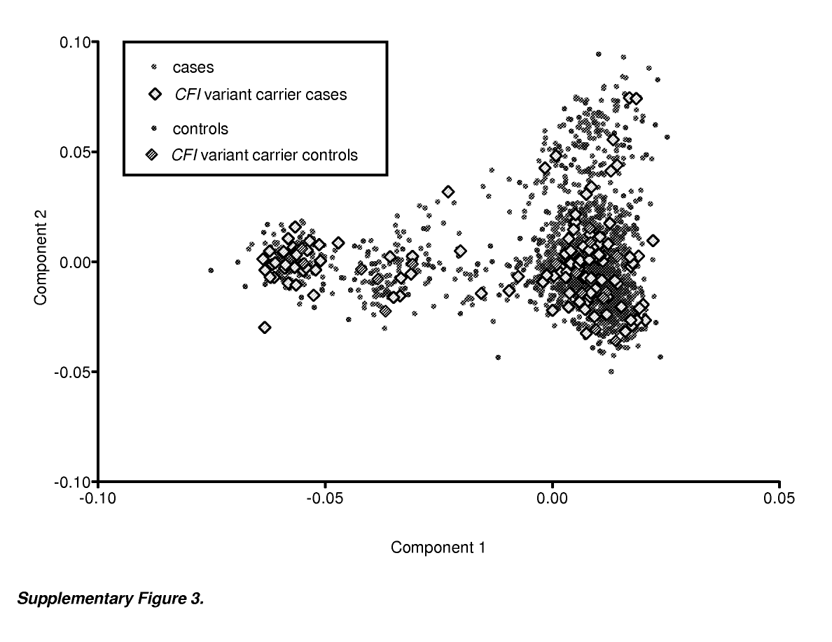

Supplementary FIG. 3 is a graphical representation of aspects of the present invention;

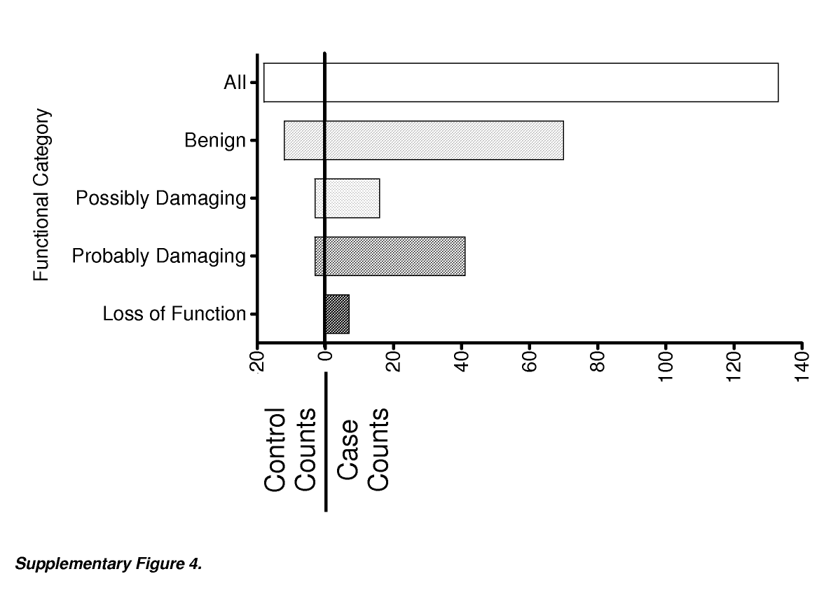

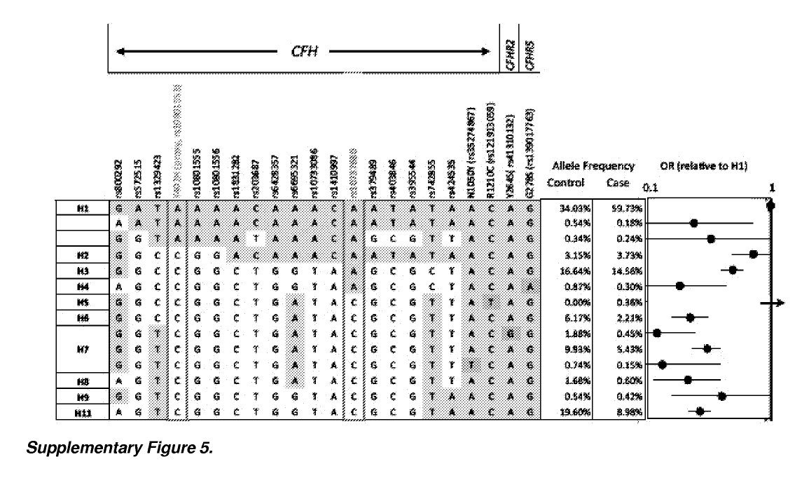

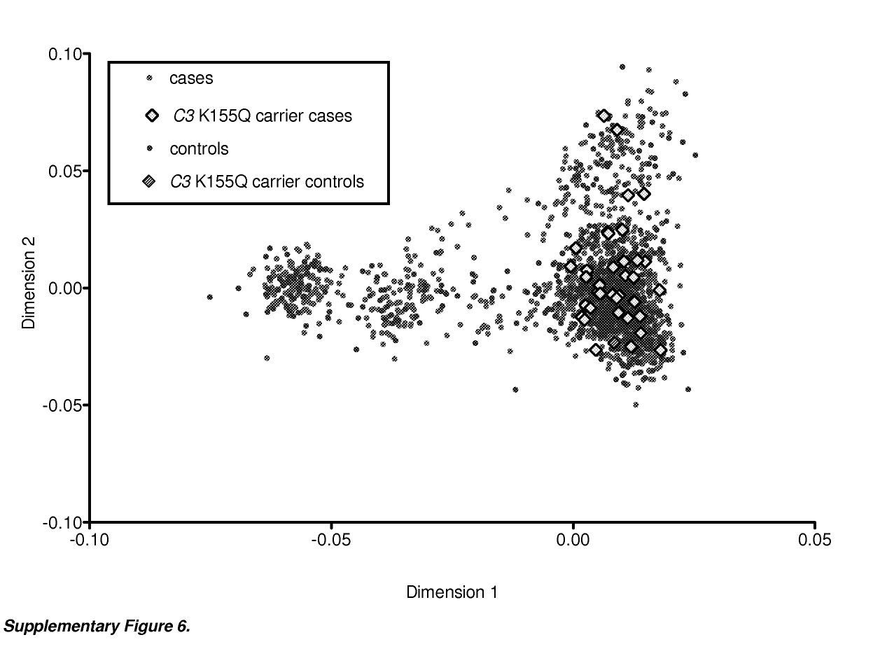

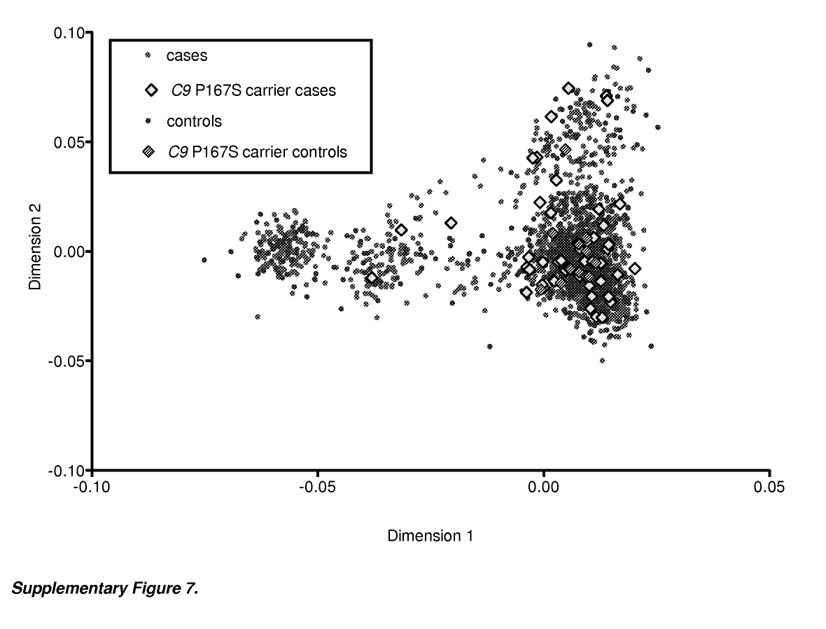

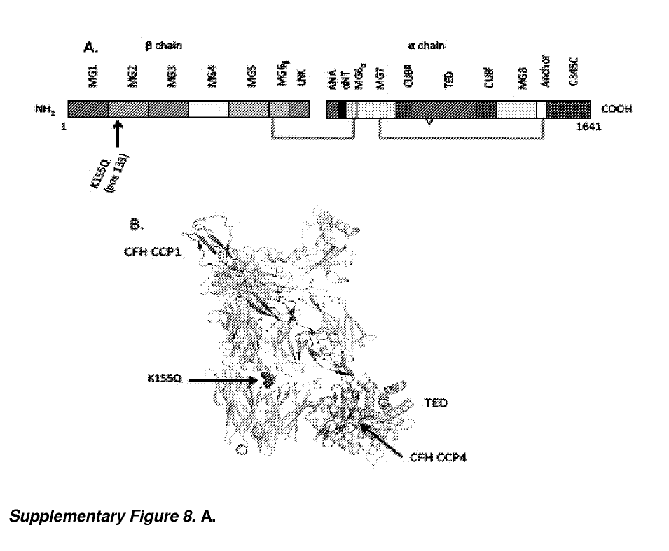

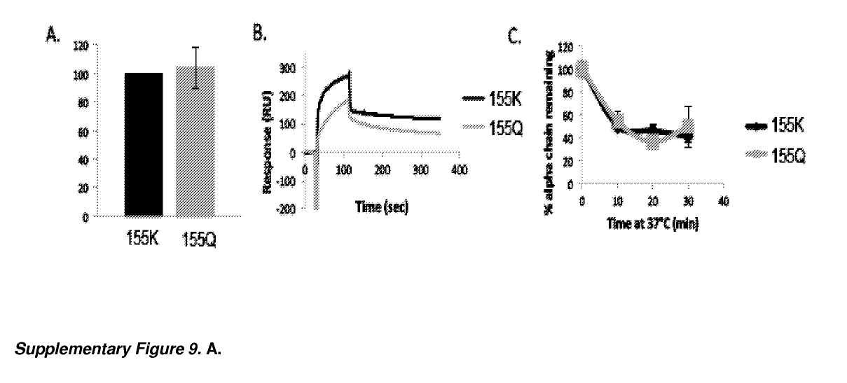

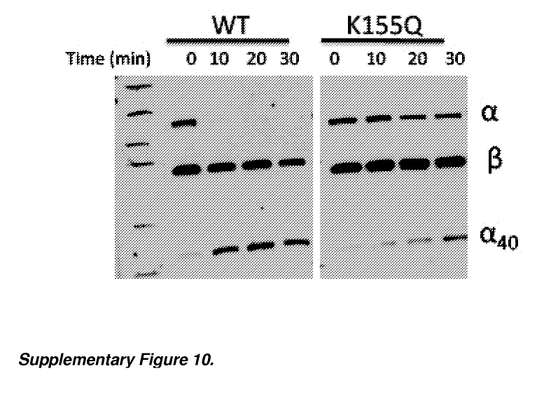

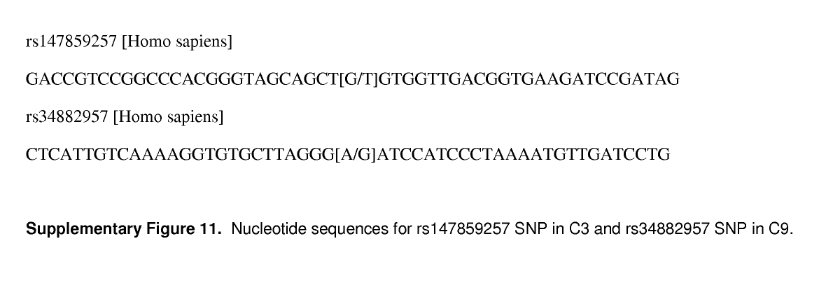

Supplementary FIG. 4 is a graphical representation of aspects of the present invention;

Supplementary FIG. 5 is a graphical representation of aspects of the present invention;

Supplementary FIG. 6 is a graphical representation of aspects of the present invention;

Supplementary FIG. 7 is a graphical representation of aspects of the present invention;

Supplementary FIG. 8 is a graphical representation of aspects of the present invention;

Supplementary FIG. 9 is a graphical representation of aspects of the present invention;

Supplementary FIG. 10 is a graphical representation of aspects of the present invention; and

Supplementary FIG. 11 is a nucleotide sequence listing.

### DETAILED DESCRIPTION

The present invention relates, in part, to the discovery that particular alleles or variants at polymorphic sites associated with genes, including complement pathway genes C3, C9, and CFI are useful as markers for AMD etiology, for determining susceptibility to AMD, and for predicting or monitoring disease progression or severity, e.g., to determine a treatment course and/or to titrate dosages of therapeutic agents. More specifically, methods are provided for determining a risk of an individual developing AMD or progressing to advanced forms of AMD (e.g., geographic atrophy and/or wet AMD) using these genetic markers. More specifically, and by non-limiting example, the single nucleotide polymorphisms (SNPs) rs147859257 in the C3 gene (p=5.2×10̂-9, OR=3.8) and rs34882957 in the C9 gene (p=6.5×10̂-7, OR=2.2) can be used as markers for AMD etiology, for determining susceptibility to AMD, and for predicting disease progression or severity, and for distinguishing risk of geographic atrophy, the advanced dry type of AMD from the advanced wet form of AMD (See Supplementary FIG. 11 for SNP nucleotide sequences for rs147859257 and rs34882957). In addition, Supplementary Tables 2 and 3 list additional polymorphisms that are also useful as such markers. Furthermore, genes and/or markers in linkage disequilibrium with these SNPs provide additional such markers.

For example, in one aspect, the invention provides a method of screening for age-related macular degeneration (AMD) in a human subject. The method can include determining a risk of AMD progression in the subject by analyzing a sample obtained from the subject for the presence in the subject's genome of at least one single nucleotide polymorphism (SNP) identified in Supplementary Tables 2 or 3, or in Table 1, or a proxy therefor. In some embodiments, a proxy is a marker that is in linkage disequilibrium with a particular SNP or marker of interest. The presence of a SNP indicates that the subject has an increased risk of developing AMD or developing an advanced form of AMD. The markers can be used individually or in combination when screening a subject. Preferred SNPs include, but are not limited to, rs147859257 (K155Q variant in C3) and rs34882957 (P167S variant in C9). In some embodiments, the presence of a particular SNP indicates the subject has an increased risk of developing AMD. In some embodiments, the presence of a particular SNP indicates the subject has an increased risk of developing an advanced form of AMD, such as geographic atrophy and/or wet AMD, which also is referred to as neovascular disease, choroidal neovascularisation (CNV), and exudative AMD.

Various techniques can be used for analyzing a sample to determine the presence of a SNP in the subject's genome. For example, in some embodiments, the method of screening can include the steps of (i) combining a nucleic acid sample from the subject with one or more polynucleotide probes capable of hybridizing selectively to a particular SNP (e.g., any SNP identified in Supplementary Tables 2 or 3 and Table 1) or gene allele, or a proxy therefor, and (ii) detecting the presence or absence of hybridization. The probes can be oligonucleotides capable of priming polynucleotide synthesis in an amplification reaction, such as PCR or real time PCR. In some embodiments, the presence of at least one SNP is determined using a microarray. In some embodiments, the presence of at least one SNP is determined using an antibody. In various embodiments, the presence of at least one SNP is determined by sequencing a portion of the patient's genome.

In various embodiments, methods are provided for determining risk of AMD or severe forms of AMD in a human patient, the method comprising detecting in a sample from the patient who is determined to be at risk for developing age-related macular degeneration due to one or more patient specific risk factors wherein the one or more patient specific risk factors is genetic, environmental/behavioral, or demographic.

In some embodiments, the patient is asymptomatic at the time of screening for AMD, and in some embodiments, the patient displays one or more AMD like symptoms at the time of screening. In some embodiments, the sample is from a patient predetermined to be at risk for AMD based on one or more patient specific factors, such as environmental/behavioral, demographic, or genetic factors. Behavioral and environmental factors include, for example, obesity, body mass index, smoking, vitamin intake, antioxidant intake, mineral intake, dietary supplement intake, use of alcohol or drugs, poor diet, a sedentary lifestyle, medical history of heart disease or other vascular disease, and medical history of kidney or liver disease. In a particular embodiment, elevated BMI is used to determine obesity. Demographic factors can include age, sex, education level, income level, marital status, occupation, religion, birth rate, death rate, average size of a family, average age at marriage. Genetic factors can include, for example, family history of AMD, presentation of AMD symptoms, and/or detection of one or more AMD risk alleles in the patient.

In some embodiments, the method includes detecting a haplotypes that includes a particular SNP (e.g., any SNP listed in Supplementary Tables 2 and 3 and Table 1).

In some embodiments, the method includes screening for a specific subtype of AMD, such as, for example, early AMD, geographic atrophy, wet AMD, neovascular disease, choroidal neovascularisation (CNV), exudative AMD, and combinations thereof.

The invention also provides, in part, a diagnostic system. The diagnostic system can include an array of polynucleotides comprising one or more of reference sequences corresponding to the SNPs identified in Supplementary Tables 2 and 3 and Table 1. The polynucleotides can include at least six or more contiguous nucleotides, and the polynucleotides can include an allelic polymorphism or SNP. The system also can include an array reader, an image processor, a database having AMD allelic data records and patient information records, a processor, and an information output. The system compiles and processes patient data and outputs information relating to the statistical probability of the patient developing AMD.

The system can be used for various methods, including contacting a subject sample or portion thereof to the diagnostic array under high stringency hybridization conditions; inputting patient information into the system; and obtaining from the system information relating to the statistical probability of the patient developing AMD.

Further provided are methods for diagnosing risk of AMD or severe forms of AMD in a human subject. The method includes combining genetic risk with behavioral risk or environmental risk, wherein the genetic risk is determined by detecting in a sample obtained from a subject the presence or absence of a single nucleotide polymorphism SNP listed in Supplementary Tables 2 and 3 and Table 1, or proxy therefor, wherein the presence of the allele is indicative of an increased risk of the subject developing AMD or a severe form of AMD.

In one embodiment, the present invention is directed to a method for determining AMD or a susceptibility to AMD in a subject comprising combining genetic risk with behavioral risk, wherein the genetic risk is determined by detecting the presence or absence of a particular allele at a polymorphic site associated with a complement pathway gene, wherein the allele is indicative of AMD or a susceptibility to AMD. In a particular embodiment, the presence or absence of a particular allele is detected by a hybridization assay. In a particular embodiment, the presence or absence of a particular allele is determined using a microarray. In a particular embodiment, the presence or absence of a particular allele is determined using an antibody. In a particular embodiment, the presence or absence of a particular allele is determined by sequencing.

As used herein, “gene” is a term used to describe a genetic element that gives rise to expression products (e.g., pre-mRNA, mRNA and polypeptides). A gene can include regulatory elements, exons and sequences that otherwise appear to have only structural features, e.g., introns and untranslated regions.

The genetic markers disclosed herein are particular “alleles” at “polymorphic sites” associated with various genes, including C3, C9, and any markers identified in Supplementary Tables 2 and 3 and Table 1. A nucleotide position at which more than one nucleotide can be present in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). If at a particular chromosomal location, for example, one member of a population has an adenine and another member of the population has a thymine at the same genomic position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

A genetic marker is “associated” with a genetic element or phenotypic trait, for example, if the marker is co-present with the genetic element or phenotypic trait at a frequency that is higher than would be predicted by random assortment of alleles (based on the allele frequencies of the particular population). Association also indicates physical association, e.g., proximity in the genome or presence in a haplotype block, of a marker and a genetic element.

A reference sequence is typically referred to for a particular genetic element, e.g., a gene. The reference sequence, often chosen as the most frequently occurring allele, is referred to as a “wild type” allele or the “major allele”). Alleles that are more common or less common in individuals with a disease/trait compared to individuals without the disease/trait, with a certain level of statistical significance, are referred to as the variant alleles. The corresponding genotype is referred to as a genetic variant.

Some variant alleles can include changes that affect a polypeptide or protein, e.g., the polypeptide encoded by a variant allele. These sequence differences, when compared to a reference nucleotide sequence, can include, for example, the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence.

Alternatively, a polymorphism associated with AMD or a susceptibility to AMD can be a synonymous change in one or more nucleotides (i.e., a change that does not result in a change to a codon of a complement pathway gene). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and -polypeptides encoded by variant alleles are referred to as “variant” polypeptides with variant amino acid sequences.

A haplotype is a combination or set of genetic markers, e.g., particular alleles at polymorphic sites, such as, e.g., SNPs and/or microsatellites. The haplotypes described herein are associated with AMD and/or a susceptibility to AMD. Detection of the presence or absence of the haplotypes herein, therefore, is indicative of AMD, is indicative of a susceptibility to AMD, is indicative of a factor related to progression from early to intermediate or late stages of AMD, is indicative of progression from intermediate to late stages of AMD, or is indicative of a lack of AMD. Detecting haplotypes, therefore, can be accomplished by methods known in the art for detecting sequences at polymorphic sites.

“Linkage” refers to a higher than expected statistical association of genotypes and/or phenotypes with each other. Linkage disequilibrium (“LD”) refers to a non-random assortment of two genetic elements. If a particular genetic element (e.g., an allele at a polymorphic site), for example, occurs in a population at a frequency of 0.25 and another occurs at a frequency of 0.25, then the predicted occurrence of a person's having both elements is 0.125, assuming a random distribution of the elements. If, however, it is discovered that the two elements occur together at a frequency higher than 0.125, then the elements are said to be in LD since they tend to be inherited together at a higher frequency than what their independent allele frequencies would predict. Roughly speaking, LD is generally correlated with the frequency of recombination events between the two elements. Allele frequencies can be determined in a population, for example, by genotyping individuals in a population and determining the occurrence of each allele in the population. For populations of diploid individuals, e.g., human populations, individuals will typically have two alleles for each genetic element (e.g., a marker or gene).

The invention is also directed to markers identified in a “haplotype block” or “LD block”. These blocks are defined either by their physical proximity to a genetic element, e.g., a C3, C9, CFI, or the other markers provided herein, or by their “genetic distance” from the element. Markers and haplotypes identified in these blocks, because of their association with AMD and C3, C9, CFI, or the markers identified herein, are encompassed by the invention. One of skill in the art will appreciate regions of chromosomes that recombine infrequently and regions of chromosomes that are “hotspots”, e.g., exhibiting frequent recombination events, are descriptive of LD blocks. Regions of infrequent recombination events bounded by hotspots will form a block that will be maintained during cell division. Thus, identification of a marker associated with a phenotype, wherein the marker is contained within an LD block, identifies the block as associated with the phenotype. Any marker identified within the block can therefore be used to indicate the phenotype.

Additional markers that are in LD with the markers of the invention or haplotypes are referred to herein as “surrogate” markers (i.e., “proxy” markers). Such a surrogate is a marker for another marker or another surrogate marker. Surrogate markers are themselves markers and are indicative of the presence of another marker, which is in turn indicative of either another marker or an associated phenotype.

Susceptibility for developing AMD includes an asymptomatic patient showing increased risk to develop AMD, and a patient having early or intermediate stages of AMD indicating a progression toward more advanced forms of AMD and expected visual loss. Susceptibility for not developing AMD includes an asymptomatic patient having at least one wild type allele, or a non-risk genotype, or a protective genotype, or a non-risk allele, or a protective allele, or a non-risk haplotype, or a protective haplotype indicates a lack of a predisposition for developing AMD.

Genetic markers (e.g., SNPs) can be detected in nucleic acids (e.g., DNA or mRNA) in any suitable sample source obtained or taken from an individual, including blood, saliva, feces, bone, epithelial cells, endothelial cells, blood cells, and other bodily fluids, cells, and/or tissues.

### Prognosis and Diagnosis of Advanced AMD.

In one aspect, the invention provides a method of determining a subject's, for example, a human subject's, risk of developing age-related macular degeneration. The method comprises determining whether the subject has a risk variant at a polymorphic site of the C3 gene, wherein, if the subject has at least one risk variant, the subject is more likely to develop age-related macular degeneration than a person without the risk variant. One exemplary risk variant is at a SNP, rs147859257 (or K155Q) in C3.

In another aspect, the invention provides a method of determining a subject's, for example, a human subject's, risk of developing age-related macular degeneration. The method comprises determining whether the subject has a risk variant at a polymorphic site of the C9 gene, wherein, if the subject has at least one risk variant, the subject is more likely to develop age-related macular degeneration than a person without the risk variant. One exemplary risk variant is at a SNP, rs34882957 (or P167S) in C9.

In one aspect, the invention provides a method of determining a subject's, for example, a human subject's, risk of developing age-related macular degeneration. The method comprises determining whether the subject has a risk or protective variant at a polymorphic site of any of the genes listed in Supplementary Tables 2 or 3, wherein, if the subject has at least one protective variant, the subject is less likely to develop age-related macular degeneration than a person without the protective variant, and wherein, if the subject has at least one risk variant, the subject is more likely to develop age-related macular degeneration than a person without the risk variant.

The presence of a protective and/or risk variant can be determined by standard nucleic acid detection assays including, for example, conventional SNP detection assays, which may include, for example, amplification-based assays, probe hybridization assays, restriction fragment length polymorphism assays, and/or direct nucleic acid sequencing.

### Diagnostic Gene Array

In one aspect, the invention comprises an array of gene fragments, particularly nucleic acids including one or more reference sequences identified in Supplementary Table 3 and Table 1 and probes for detecting the allele at the SNPs of one or more reference sequences identified in Supplementary Tables 2 or 3 and Table 1. Polynucleotide arrays provide a high throughput technique that can assay a large number of polynucleotide sequences in a single sample. This technology can be used, for example, as a diagnostic tool to assess the risk potential of developing AMD using the SNPs and probes of the invention. Polynucleotide arrays (for example, DNA or RNA arrays), include regions of usually different sequence polynucleotides arranged in a predetermined configuration on a substrate, at defined x and y coordinates. These regions (sometimes referenced as “features”) are positioned at respective locations (“addresses”) on the substrate. The arrays, when exposed to a sample, will exhibit an observed binding pattern. This binding pattern can be detected upon interrogating the array. For example, all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Arrays can be fabricated by depositing previously obtained biopolymers onto a substrate, or by in situ synthesis methods. The substrate can be any supporting material to which polynucleotide probes can be attached, including but not limited to glass, nitrocellulose, silicon, and nylon. Polynucleotides can be bound to the substrate by either covalent bonds or by non-specific interactions, such as hydrophobic interactions. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. Further details of fabricating biopolymer arrays are described in U.S. Pat. No. 6,242,266; U.S. Pat. No. 6,232,072; U.S. Pat. No. 6,180,351; U.S. Pat. No. 6,171,797; EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Other techniques for fabricating biopolymer arrays include known light directed synthesis techniques. Commercially available polynucleotide arrays, such as Affymetrix GeneChip™, can also be used. Use of the GeneChip™, to detect gene expression is described, for example, in Lockhart et al., Nat. Biotechnol., 14: 1675, 1996; Chee et al., Science, 274:610, 1996; Hacia et al, Nat. Gen., 14:441, 1996; and Kozal et al., Nat. Med., 2:753, 1996. Other types of arrays are known in the art, and are sufficient for developing an AMD diagnostic array of the present invention.

To create the arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences including the SNPs identified in Supplementary Table 3 and Table 1, or the complement thereof. Preferred arrays comprise at least one single-stranded polynucleotide probe comprising at least 6, 7, 8, 9, 10, 11, 12, 13, 14, 15, 16, 17, 18, 19, 20, 25, or 30 or more contiguous nucleotides selected from the nucleotide sequences including the SNPs identified in Supplementary Tables 2 or 3, or Table 1, or the complement thereof.

Tissue samples from a subject can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels that can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance. Preferably, after the array has been exposed to a sample, the array is read with a reading apparatus (such as an array “scanner”) that detects the signals (such as a fluorescence pattern) from the array features. Such a reader preferably would have a very fine resolution (for example, in the range of five to twenty microns) for an array .having closely spaced features.

The signal image resulting from reading the array can then be digitally processed to evaluate which regions (pixels) of read data belong to a given feature as well as to calculate the total signal strength associated with each of the features. The foregoing steps, separately or collectively, are referred to as “feature extraction” (U.S. Pat. No. 7,206,438). Using any of the feature extraction techniques so described, detection of hybridization of a patient derived polynucleotide sample with one of the AMD markers on the array given as the nucleotide sequences including the SNPs identified in Supplementary Tables 2 or 3, or Table 1 identifies that subject as having or not having a genetic risk factor for AMD, as described above.

### System for Analyzing Patient Data

In another aspect, the invention provides a system for compiling and processing patient data, and presenting a risk profile for developing AMD or for the progression to late stages. A computer aided medical data exchange system is preferred. The system is designed to provide high-quality medical care to a patient by facilitating the management of data available to care providers. The care providers will typically include physicians, surgeons, nurses, clinicians, various specialists, and so forth. It should be noted, however, that while general reference is made to a clinician in the present context, the care providers may also include clerical staff, insurance companies, teachers and students, and so forth. The system provides an interface, which allows the clinicians to exchange data with a data processing system. The data processing system is linked to an integrated knowledge base and a database.

The database may be software-based, and includes data access tools for drawing information from the various resources as described below, or coordinating or translating the access of such information. In general, the database will unify raw data into a useable form. Any suitable form may be employed, and multiple forms may be employed, where desired, including hypertext markup language (HTML) extended markup language (XML), Digital Imaging and Communications in Medicine (DICOM), Health Level Seven™ (HL7), and so forth. In the present context, the integrated knowledge base is considered to include any and all types of available medical data that can be processed by the data processing system and made available to the clinicians for providing the desired medical care. In general, data within the resources and knowledge base are digitized and stored to make the data available for extraction and analysis by the database and the data processing system. Even where more conventional data gathering resources are employed, the data is placed in a form that permits it to be identified and manipulated in the various types of analyses performed by the data processing system.

The integrated knowledge base is intended to include one or more repositories of medical-related data in a broad sense, as well as interfaces and translators between the repositories, and processing capabilities for carrying out desired operations on the data, including analysis, diagnosis, reporting, display and other functions. The data itself may relate to patient-specific characteristics as well as to non-patient specific information, as for classes of persons, machines, systems and so forth. Moreover, the repositories may include devoted systems for storing the data, or memory devices that are part of disparate systems, such as imaging systems. As noted above, the repositories and processing resources making up the integrated knowledge base may be expandable and may be physically resident at any number of locations, typically linked by dedicated or open network links. Furthermore, the data contained in the integrated knowledge base may include both clinical data (e.g., data relating specifically to a patient condition) and non-clinical data. Examples of preferred clinical data include patient medical histories, patient serum, plasma, and/or other biomarkers such as blood levels of certain other nutrients, fats, female and male hormones, etc., and cellular antioxidant levels, and the identification of past or current environmental, lifestyle and other factors that predispose a patient to develop AMD. These include but are not limited to various risk factors such as obesity, smoking, vitamin and dietary supplement intake, use of alcohol or drugs, poor diet, a sedentary lifestyle, medical history of heart disease or other vascular disease, and/or medical history of kidney or liver disease. Non-clinical data may include more general information about the patient, such as residential address, data relating to an insurance carrier, and names and addresses or phone numbers of significant or recent practitioners who have seen or cared for the patient, including primary care physicians, specialists, and so forth.

The flow of information can include a wide range of types and vehicles for information exchange. In general, the patient can interface with clinicians through conventional clinical visits, as well as remotely by telephone, electronic mail, forms, and so forth. The patient can also interact with elements of the resources via a range of patient data acquisition interfaces that can include conventional patient history forms, interfaces for imaging systems, systems for collecting and analyzing tissue samples, body fluids, and so forth. Interaction between the clinicians and the interface can take any suitable form, depending upon the nature of the interface. Thus, the clinicians can interact with the data processing system through conventional input devices such as keyboards, computer mice, touch screens, portable or remote input and reporting devices. The links between the interface, data processing system, the knowledge base, the database and the resources typically include computer data exchange interconnections, network connections, local area networks, wide area networks, dedicated networks, virtual private network, and so forth.

In general, the resources can be patient-specific or patient-related, that is, collected from direct access either physically or remotely (e.g., via computer link) from a patient. The resource data can also be population-specific so as to permit analysis of specific patient risks and conditions based upon comparisons to known population characteristics. It should be noted that the resources can generally be thought of as processes for generating data. While many of the systems and resources will themselves contain data, these resources are controllable and can be prescribed to the extent that they can be used to generate data as needed for appropriate treatment of the patient. Exemplary controllable and prescribable resources include, for example, a variety of data collection systems designed to detect physiological parameters of patients based upon sensed signals. Such electrical resources can include, for example, electroencephalography resources (EEG), electrocardiography resources (ECG), electromyography resources (EMG), electrical impedance tomography resources (EIT), nerve conduction test resources, electronystagmography resources (ENG), and combinations of such resources. Various imaging resources also can be controlled and prescribed as necessary. Exemplary eye tests include, for example, electrophysiologic tests, elcetroretinograms, electrooculagrams, retinal angiography, retinal photography, ultrasonography, optical coherence tomography, and other imaging modalities such as autofluorescence. A number of modalities of such resources are currently available, such as, for example, X-ray imaging systems, magnetic resonance (MR) imaging systems, computed tomography (CT) imaging systems, positron emission tomography (PET) systems, fluorography systems, sonography systems, infrared imaging systems, nuclear imaging systems, thermoacoustic systems, and so forth. Imaging systems can draw information from other imaging systems, electrical resources can interface with imaging systems for direct exchange of information (such as for timing or coordination of image data generation, and so forth).

In addition to such electrical and highly automated systems, various resources of a clinical and laboratory nature can be accessible. Such resources may include blood, urine, saliva and other fluid analysis resources, including gastrointestinal, reproductive, urological, nephrological (kidney function), and cerebrospinal fluid analysis system. Such resources can further include polymerase (PCR) chain reaction analysis systems, genetic marker analysis systems, radioimmunoassay systems, chromatography and similar chemical analysis systems, receptor assay systems and combinations of such systems. Histologic resources, somewhat similarly, can be included, such as tissue analysis systems, cytology and tissue typing systems and so forth. Other histologic resources can include immunocytochemistry and histopathological analysis systems. Similarly, electron and other microscopy systems, in situ hybridization systems, and so forth can constitute the exemplary histologic resources. Pharmacokinetic resources can include such systems as therapeutic drug monitoring systems, receptor characterization and measurement systems, and so forth. Again, while such data exchange can be thought of passing through the data processing system, direct exchange between the various resources can also be implemented.

Use of the present system involves a clinician obtaining a patient sample, and evaluation of the presence of a genetic marker in that patient indicating a predisposition (or not) for AMD or its progression, such as one or more of the nucleotide sequences including the SNPs identified in Supplementary Table 3 and Table 1 alone or in combination with other known risk factors. The clinician or their assistant also obtains appropriate clinical and non-clinical patient information, and inputs it into the system. The system then compiles and processes the data, and provides output information that includes a risk profile for the patient, of developing AMD and/or progressing to advanced forms of AMD.

The present invention thus provides for certain polynucleotide sequences that have been correlated to AMD. These polynucleotides are useful as diagnostics, and are preferably used to fabricate an array, useful for screening patient samples. The array, in a currently most preferred embodiment, is used as part of a laboratory information management system, to store and process additional patient information in addition to the patient's genomic profile. As described herein, the system provides an assessment of the patient's risk for developing AMD, risk for disease progression, and likelihood of disease prevention based on patient controllable factors.

### Kits

The invention relates in part to kits and systems useful for performing the diagnostic methods described herein. The methods described herein can be performed by, for example, diagnostic laboratories, service providers, experimental laboratories, and individuals. The kits can be useful in these settings, among others.

Kits include reagents and materials for obtaining genetic material and assaying one or more markers in a sample from an individual, analyzing the results, diagnosing whether the individual is susceptible to or at risk for developing AMD, monitoring disease progression, and/or determining an appropriate treatment course. For example, in some embodiments, the kit can include a needle, syringe, vial, cotton swap or other apparatus for obtaining and/or containing a sample from an individual. In some embodiments, the kit can include at least one reagent which is used specifically to detect a marker disclosed herein. That is, suitable reagents and techniques readily can be selected by one of skill in the art for inclusion in a kit for detecting or quantifying a marker of interest.

For example, where the marker is a nucleic acid (e.g., DNA or RNA), the kit includes reagents appropriate for detecting nucleic acids using, for example, PCR, hybridization techniques, and microarrays.

Where appropriate, the kit includes: extraction buffers or reagents, amplification buffers or reagents, reaction buffers or reagents, hybridization buffers or reagents, immunodetection buffers or reagents, labeling buffers or reagents, and detection means. The kit can include all or part of the nucleic acids of the nucleotide sequences including the SNPs identified in Supplementary Table 3 and Table 1, or a nucleic acid molecule complementary thereto.

Kits can also include a control, which can be a control sample, a reference sample, an internal standard, or previously generated empirical data. The control may correspond to a known allele, e.g., a wild type and/or a variant allele. In addition, a control may be provided for each marker or the control may be a reference (e.g., a wild type and/or variant sequence).

Kits can include one or more containers for each individual reagent. Kits can further include instructions for performing the methods described herein and/or interpreting the results, in accordance with any regulatory requirements. In addition, software can be included in the kit for analyzing the results. Preferably, the kits are packaged in a container suitable for commercial distribution, sale, and/or use.

The following examples are provided for illustration, not limitation.

### EXAMPLES

### Example 1

To define the role of rare variants in advanced age-related macular degeneration (AMD) risk, we sequenced exons of 779 genes within AMD loci and pathways in 2,493 cases and controls. We found that 7.8% of AMD cases compared to 2.3% of controls are carriers of rare missense CFI variants (OR=3.6, p=2×10⁻⁸); some of these variants result in loss of CFI function. We also observed significant association in rare missense alleles outside of CFI. Genotyping in 5,115 independent samples confirmed associations to AMD with a K155Q allele in C3 (joint p=5.2×10⁻⁹, OR=3.8) and a P167S allele in C9 (joint p=6.5×10⁻⁷, OR=2.2). Finally, we show that the 155Q allele reduces C3 binding to CFH in vitro, mitigating subsequent deactivation of C3 by CFI cleavage. These results implicate loss of C3 protein regulation by CFH and CFI leading to excessive alternative complement activation in AMD pathogenesis, thus informing both the direction of effect and mechanistic underpinnings of this disorder.

Age-related macular degeneration (AMD) is a common progressive disease that can lead to irreversible vision loss in individuals over 55 years of age with genetic risk factors¹⁻³. Most genetic variants identified for AMD are common, without clearly established disease mechanisms⁴. To date, highly penetrant missense variants that confer AMD risk have been discovered only in CFH^{5,6}. We sought to identify additional rare missense variants that contribute to AMD risk, with the broader goal of identifying specific disease mechanisms and also elucidating the direction of effect of such alleles. Such data could have a direct impact on the design of therapeutic paradigms.

We applied next-generation targeted sequencing to exons, 5′ untranslated regions, and 3′ untranslated regions for 779 candidate genes in known AMD loci and in pathways related to AMD pathogenesis (see Online Methods). In total we sequenced 5.28 megabases in 1,676 cases, 745 controls, and 36 siblings with discordant disease status (n=2,493), obtaining>20× coverage for a median of 95.6% of the targeted region (Supplementary FIG. 1). After calling variants with GATK⁷ and applying strict quality control, our data had <0.92% missing genotypes for each allele, and <0.54% missing genotypes for each individual. Genotypes for close proxies to known AMD risk variants were captured in our sequencing experiment had effect sizes comparable to published values (Supplementary Table 1).

To assess the accuracy of genotypes obtained by sequencing we compared them to genotype data for the same individuals obtained by Illumina HumanExome BeadChip™ array (i.e. exome-chip)^{8,9}. We observed 99.97% concordance for 2,426 sequenced missense SNPs with allele frequencies of at least 0.001 that were on the exome-chip (Online Methods). Allelic dosages were almost perfectly correlated (r>0.99) for 96.5% of common variants (f>0.01) and 93.0% of rare variants (Supplementary FIG. 2). Indeed, only 0.2% and 3.6% of common and rare SNPs, respectively had modestly correlated dosages (r<0.9).

We next used our sequencing data to test whether any individual gene had a higher burden of rare variants in cases or in controls. For these analyses we selected only rare variants (f<0.01) that alter coding sequences (missense, nonsense, read-through, or splice variants, n=18,854 SNPs). We used a simple burden test to assess whether the proportion of case individuals who carried at least one rare variant was in excess of chance (Online Methods)¹⁰. We similarly tested increased burden in controls. We saw a significantly increased burden of rare variants in cases in only one gene, CFI (exact one-tailed p=1.6×10⁻⁸, OR=3.57, FIG. 1A). In the sequencing data, 7.8% of cases had a rare coding variant, compared to 2.3% of controls.

The enrichment of rare coding variants in CFI is not likely to be the consequence of population stratification. First, if stratification were driving the observed enrichment, then other genes would also demonstrate enrichment for rare variants; however burden tests for other genes did not exceed expectation due to chance (FIG. 1A). Second, stratification would also drive enrichment of synonymous variants. But, we observed no case enrichment for the 12 rare synonymous variants in CFI (p=0.85). Third, stratification could be assessed and adjusted for with principal components based on ancestry informative markers. We calculated principal components on a subset of 1,558 cases and 683 controls for which we had exome-chip data. We found no obvious clustering of CFI rare variant carrier samples along the top two principal components (Supplementary FIG. 3). Furthermore, we observed no change in enrichment of rare CFI coding variants in cases when we controlled for ancestry by applying logistic regression adjusting for the top five principal components (p=5.0×10⁻⁸, OR=3.6, compared to p=1.1×10⁻⁷, OR=3.5 after adjusting).

The case-enrichment of rare coding variants in CFI was independent of the nearby common risk allele, rs4698775^{4,11}. Stratifying our samples for rs4698775 genotypes did not obviate the CFI rare coding variant enrichment (p=1.7×10⁻⁸, FIG. 1B); in fact, individuals with any rs4698775 genotype had a similar degree of CFI rare variant enrichment. Also, the risk conferred by the common rs4698775 allele was independent of rare variant carrier status (p<0.04, allelic OR=1.15), arguing against a “synthetic association”.

We examined the 59 separate CFI nucleotide variants that conferred 58 coding changes (FIG. 2, Supplementary Table 2). We used PolyPhen-2¹² to classify variants into four categories based on their potential predicted impact (1) loss of function (nonsense or splice variants)¹³, (2) probably damaging, (3) possibly damaging and (4) benign. We observed that loss of protein function variants (three nonsense and one splice variant) were the most skewed toward cases (7 to O, Supplementary FIG. 4). Probably damaging variants were more skewed towards cases (41 cases to 3 controls) than possible damaging variants (16 to 3) and benign variants (70 to 12). Indeed, probably damaging or loss of function variants conferred high risk of disease (OR=7.5, p=1.3×10⁻⁵). The CFI gene encodes complement factor I, which can be divided into a light chain, containing the catalytic serine protease domain, responsible for proteolytically cleaving the C3 protein, and a heavy chain. There is a greater burden of rare variants in the catalytic light chain (OR=4.85, p=2.2×10⁻⁶) than in the heavy chain (OR=2.63, p=0.0012). CFI had no common variants (f>0.01) that altered the coding sequence. None of the individual variants in CFI were specifically associated with AMD (p>0.02), which may be due to their low frequency.

Remarkably, many of these CFI rare variants have been seen in atypical hemolytic uremic syndrome cases (aHUS), including P50A¹⁴, G119R¹⁴, A240G¹⁵, G261D¹⁶, R317W¹⁵, I340T¹⁷, Y369S^{15,18}, D403N¹⁴, 1416L¹⁸, Y459S¹⁴, R474X¹⁹, and P553S¹⁴. These variants are hypothesized to confer aHUS risk by decreasing the ability of CFI to cleave and thereby deactivate C3b, the cleaved and activated form of C3. Of these variants R317W and 1340T result in CFI functional deficiency²⁰, while P50A, 1416L, and R474X produced in a CFI protein quantitative deficiency¹⁴. The G261D variant has been studied extensively and no functional deficiency has been noted to date¹⁶.

We then used the same sequencing data to test rare variants individually for association to AMD risk. This study was not powered to detect an association for variants observed less than five times and we therefore excluded those extremely rare SNPs from this test. Overall, we identified 2,169 variants that had an allele frequency of <1% in cases or controls and passed strict quality control (Online Methods). Of these, we identified five and 16 variants associated with increased and decreased AMD risk respectively (exact 1-tailed p<0.01, Supplementary Table 3). Four of these variants were within or near CFH, including the previously reported CFH R1210C risk variant (p=0.0012)⁵. In addition we observed association to the CFH N1050Y, CFHR2Y264C, and CFHR5G278S protective variants. After phasing common variants in samples from the sequencing experiment within this locus, we observed that these variants were in LD with CFH haplotypes (Supplementary FIG. 6). We could not be certain whether these associations represented unique effects or were the consequence of tagging CFH haplotypes with large effects, potentially driven by effects from Y402H and the rs10737680 intronic alleles.

We evaluated 11 of the 17 variants outside of the CFH locus for evidence of association in 2,227 separate cases and 2,888 separate controls from Boston, Baltimore, and France using either the exome-chip or Taqman (Supplementary Table 4). Of those variants, we observed independent evidence of association for two variants in replication (p<0.0045=0.05/11, see Table 1).

The two variants that ultimately demonstrated association in replication were a nonsynonymous rs147859257 (or K155Q) variant in C3 (exact 1-tailed p=4.8×10⁻⁶ in discovery) and the rs34882957 (P167S) variant in C9 (p=2.3×10⁻³ in discovery, Table 1, Supplementary Table 3). Both variants had highly concordant sequence-based genotypes compared to separate exome-chip genotypes (r=1). To assess the possibility that either association could be related to population stratification, we calculated principal components for a subset of samples (n=2,241) as described above for CFI variants. There was no evidence of clustering of individuals that were carriers of either of these variants along the first two principal components (Supplementary FIGS. 6 and 7). When controlling for ancestry by adjusting for the top five principal components, we observed no change in the K155Q effect (p=2.7×10⁻⁵, OR=16.1 compared to p=2.0×10⁻⁶, OR=16.7, after adjusting). Or in the P167S effect (p=0.053, OR=2.0 compared to p=0.053, OR=2.0, after adjusting).

The C3 K155Q variant, conferring a lysine to glutamine change at position 155 (or position 133 in the mature protein excluding the signal peptide), demonstrated compelling evidence of association in replication (p=3.5×10⁻⁵) and was highly significant with a large effect size in joint analysis with discovery samples (p=5.2×10⁻⁹, OR=3.8; Table 1A). This risk is independent from a previously discovered common risk variant, R102G (rs2230199)^{21,22}. In the samples with genotypes for both R102G and K155Q (n=6,935), we observed increased significance of the risk conferred by K155Q when we stratified individuals on R102G genotypes (from p=5.4×10⁻⁹ to p=6.2×10⁻¹⁰, Table 1A). We found that the K155Q risk variant is, in fact, in phase with the protective 102R (rs2230199-G/102-R) allele. The C9 P167S variant also demonstrated convincing evidence of association in replication (p=2.4×10⁻⁵) and in joint analysis (p=6.5×10⁻⁷, OR=2.2; Table 1B). While nominal associations have been reported previously²³, our result implicates C9 in AMD pathogenesis definitively for the first time. This expands the repertoire of AMD genes in the complement cascade, specifically implicating the membrane attack complex (C5-C9) formed downstream of alternative complement pathway activation.

Since K155Q is exposed on the protein surface of the C3 β-chain in a positively charged patch, close to the CFH binding site (Supplementary FIG. 8)²⁴, we hypothesized that it might disrupt CFH binding. We applied an experimental strategy that we previously devised to evaluate aHUS mutations in C3²⁵. We engineered wild type (155K) cDNA to express a C3 protein encoding the 155Q amino acid and assessed CFH binding to C3 with an enzyme-linked immunosorbent assay (ELISA) assay. The 155Q protein demonstrated ˜20% lower binding to CFH compared to 155K (FIG. 3A), but had normal binding to two other complement regulators, MCP and CR1 (Supplementary FIG. 9A), arguing that the observed biochemical phenotype is specific. In surface plasmon resonance experiments, the 155Q protein had significantly decreased binding to both CFH (FIG. 3B) and MCP (Supplementary FIG. 9B). Since CFH serves as a cofactor for CFI mediated cleavage and deactivation of C3, we hypothesized that in the presence of 155Q, C3 would be resistant to CFI-mediated inactivation. We observed substantially decreased cleavage of 155Q compared to 155K C3 at all time points in fluid phase cofactor assays (FIG. 3C, Supplementary FIG. 10). However, similar assays with MCP did not show a difference in cofactor activity (Supplementary FIG. 9C), similarly arguing that the effect of the K155Q variant is specific to CFH binding.

We conclude that C3 K155Q results in impaired binding of the C3b active form to CFH and subsequent inactivation by CFI, resulting in increased C3 convertase and feedback amplification of the alternative pathway (FIG. 4). The effect of K155Q mirrors that of the highly penetrant R1210C substitution in CFH, which also disrupts CFH binding to C3b²⁶⁻²⁸. The high penetrance of both variants implicates a critical role for alternative pathway regulation by the joint effect of CFH and CFI in inactivating C3b. The increased burden of CFI mutations, particularly the loss of function mutations in the catalytic domain of the protein, adds additional support for the key role of alternative pathway regulation by this C3b-CFH-CFI tri-molecular interaction. These results also speak to the potential of therapeutic agents that either inhibit C3b, or enhance CFI or CFH, to reduce the risk or progression of age-related macular degeneration.

### Tables

TABLE 1
C3 K155Q (A) and C9 P167S (B) associations across sample collections. Each row represents association statistics for
the sample collections. The first row (Boston, sequencing) represents our discovery data set, and lists genotype counts
in the case-control samples and 49 discordant sibling pairs, and the exact one-tailed association p-values. For C3 K155Q
we list one-tailed p-values with and without conditioning for the rs2230199 R102G common variant. Other rows list similar
statistics for each of the replication cohorts. The last row represents the joint analysis of all samples.
A. C3 K155Q (rs147859257) association with AMD Risk

| | Case/Control | | | | | | | | | |
|---|---|---|---|---|---|---|---|---|---|---|
| | K155Q | | K155 | | | Discordant Sibpairs | | | K155Q Association | |
| | Heterozygotes | | Homozygotes | | | | K155Q in | K155Q in | One-tailed p-value | |
| | Un- | | Un- | | Allelic | Concordant | affected | unaffected | Un- | Conditioning |
| Cohort | affected | AMD | affected | AMD | OR | Genotypes | sib | sib | conditional | on rs2230199 |
| Boston | 1 | 40 | 744 | 1636 | 18.0 (2.5-130.9) | 36 | 0 | 0 | 4.8 × 10⁻⁶ | 9.6 × 10⁻⁶ |
| (sequencing, | | | | | | | | | | |
| discovery) | | | | | | | | | | |
| Boston | 19 | 20 | 2014 | 726 | 2.9 (1.5-5.4) | 13 | 0 | 0 | 9.3 × 10⁻⁴ | 3.6 × 10⁻⁴ |
| (replication) | | | | | | | | | | |
| French | 4 | 18 | 676 | 935 | 3.2 (1.1-9.6) | — | — | — | 0.018 | 0.011 |
| Baltimore | 3 | 17 | 163 | 467 | 2.0 (0.6-6.7) | — | — | — | 0.24 | — |
| All | 26 | 55 | 2853 | 2128 | 2.8 (1.7-4.6) | 13 | 0 | 0 | 3.5 × 10⁻⁵ | 9.2 × 10⁻⁶ |
| Replication | | | | | | | | | | |
| Joint | 27 | 95 | 3597 | 3764 | 3.8 (2.3-6.1) | 49 | 0 | 0 | 5.2 × 10⁻⁹ | 6.2 × 10⁻¹⁰ |

B. C9 P167S (rs34882957) association with AMD Risk

| | Case/Control | | | | | | | | |
|---|---|---|---|---|---|---|---|---|---|
| | P167S | | P167S | | | Discordant Sibpairs | | | |
| | Heterozygotes | | Homozygotes | | | | P167S in | P167S in | P167S Association |
| | Un- | | Un- | | Allelic | Concordant | affected | unaffected | One-tailed p-value |
| Cohort | affected | AMD | affected | AMD | OR | Genotypes | sib | sib | Un-conditional |
| Boston | 11 | 53 | 734 | 1623 | 2.2 (1.1-4.1) | 35 | 1 | 0 | 0.0068 |
| (sequencing, | | | | | | | | | |
| discovery) | | | | | | | | | |
| Boston | 39 | 29 | 1994 | 717 | 2.0 (1.3-3.3) | 12 | 1 | 0 | 0.0023 |
| (replication) | | | | | | | | | |
| French | 9 | 33 | 671 | 919 | 2.6 (1.3-5.6) | — | — | — | 0.0045 |
| Baltimore | 4 | 21 | 127 | 438 | 1.7 (0.6-5.1) | — | — | — | 0.22 |
| All | 51 | 79 | 2828 | 2128 | 2.2 (1.5-3.2) | 12 | 0 | 0 | 2.4 × 10⁻⁵ |
| Replication | | | | | | | | | |
| Joint | 63 | 136 | 3546 | 3697 | 2.2 (1.6-3.0) | 47 | 2 | 0 | 6.5 × 10⁻⁷ |

### Online Methods

### Study Sample Descriptions

Case Definitions.

Board certified ophthalmologists evaluated all case and matched (non-shared) control individuals. We either (1) clinically evaluated with visual acuity measurements, dilated slit lamp biomicroscopy and stereoscopic color fundus photography or (2) reviewed full ophthalmologic medical records and images. Case patients had either geographic atrophy (advanced dry AMD) or neovascular disease (wet AMD) (Clinical Age-Related Maculopathy Grading System (CARMS) stages 4 and 5)²⁹. Controls were also examined and had no signs of intermediate or advanced macular degeneration in either eye and absence of bilateral early AMD. All Boston and France controls and most Baltimore controls (>80%) were ≧60 years old.

Boston.

Subjects were recruited through ongoing AMD study protocols^{2,11,30-33}. We genotyped a sub-set of these samples using the Affymetrix SNP 6.0 GeneChip³⁴. These samples included 2,422 unrelated cases and 1,287 unrelated controls, and also 49 discordant sib-pairs. We genotyped all samples with the Illumina HumanExome genotyping array (see below). We selected a subset of these samples for targeted sequencing including 1,676 unrelated cases and 745 unrelated controls, as well as 36 siblings with discordant case status (see below).

France.

We recruited AMD cases and controls at the Hopital Intercommunal de Creteil (FR-CRET), Creteil, France, as previously described^{34,35}, who were self-described white individuals of European descent. These samples included 953 unrelated cases and 203 unrelated controls. We genotyped all samples with the Illumina HumanExome genotyping array.

Baltimore.

Unrelated subjects were recruited at Johns Hopkins Hospital in Baltimore, Md. as previously described^{34,36-38}, and were self-described white individuals of European descent. We genotyped these 516 cases and 163 controls for selected SNPs with TaqMan (see below).

Shared Controls.

We also augmented our samples by utilizing a collection of shared controls, that were the controls recruited for four separate studies. These samples had been broadly consented for medical use, and had been genotyped at the Broad Institute as a part of ongoing studies. In aggregate, these samples included 2,466 samples that were not evaluated for retinal diseases, Caucasian, and unrelated samples. The samples included control individuals recruited for the 1000 genomes project (n=448)³⁹, the international Serious Adverse Event Consortium (iSAEC, n=709)⁴°, the National Institutes of Mental Health controls (n=1,054)⁴¹, and the Prospective Registry in IBD Study at MGH (Prism, n=255)⁴².

### Targeted Sequencing

Gene Selection.

To identify a set of genes that were most likely to harbor rare variants, we first selected all genes within 19 genomic regions defined by common SNPs which have been associated with AMD and obtained genome-wide significance in a recent large meta-analysis of data from 18 research groups⁴. Additionally, we selected genes closest to any common SNP with nominal significance (p<10⁻⁴) in a smaller previous meta-analysis³⁵. We also selected genes involved in pathways believed to play a critical role in AMD or other retinal diseases, including complement genes, angiogenesis genes, genes involved in the structure of retinal pigment epithelium (RPE) and photoreceptors, HDL metabolism genes, genes involved in inflammation and oxidation pathways, and genes in the extracellular and collagen matrix pathways. We also included genes previously reported to be associated with AMD and related diseases, including Stargardt disease, Best disease or vitelliform macular dystrophy, Alzheimer's disease, and atypical hemolytic uremic syndrome (aHUS).

Targets Capture and Re-Sequencing.

We conducted at PerkinElmer, Inc., according to the manufacturer's protocols. Briefly, we designed a custom Agilent SureSelectXT Kit to capture genomic sequences of coding exons, splice junctions, 5′ UTR and 3′ UTR regions in 779 selected genes with indexing barcodes for each sample. We sequenced a total target length of 5.28 Mb including 1.76 MB of coding exons. We isolated the hybridized library fragments, quantitated by qPCR and sequenced, and then sequenced paired-end reads with the IIlumina HiSeq2000™ sequencing platform. We required sequencing data for each sample to have over 10× coverage at greater than 90% targeted regions and over 20× coverage at greater than 80% targeted regions. We resequenced a small fraction (<5%) of samples that didn't achieve this high quality standard.

Read Mapping, Variant Detection and Annotation.

We aligned sequence reads in each individual to the human reference genome (NCBI build 37.3, hg19) using Burrows-Wheeler Aligner (BWA, v0.59)⁴³. We called the consensus genotypes in the target regions with The Genome Analysis Toolkit (GATK, v2.18) with the workflow and parameters recommended in the best practice variant detection with GATK v4^{7,44}. Briefly, we applied GATK duplicate removal, indel realignment, base quality score recalibration, and performed multi-sample SNP and indel discovery and genotyping across all samples simultaneously using variant quality score recalibration (VQSR). Other than high quality variants assigned “PASS” by VQSR, we also included only those variants in lower tranches with truth sensitivity between 99.0-100 that were also separately recorded in the exome sequencing project (ESP) database of 6500 samples⁴⁵. We annotated variants with snpEff (v2.05)⁴⁶.

Quality Control.

We further excluded SNPs failing Hardy-Weinberg test (p<10⁻⁶). We also excluded alleles from further analysis that had high missing genotype data (>1%), likely due to systematic low coverage or difficulty mapping reads across a high proportion of samples. We also excluded samples with high missing genotype data (>1%) for common alleles with >1% frequency in our data set.

### Array-Based Genotyping of Coding Variants

We genotyped the France and Boston sample sets with the Infinium HumanExome BeadChip (v1.0), which provides coverage of over 240,000 functional exonic variants selected from >12,000 whole exome and whole genome sequences. In addition, we customized our assay by adding 3,214 SNPs in candidate AMD genes. We conducted genotyping at the John Hopkins Genotyping Core Laboratory. We genotyped shared control samples separately at the Broad Institute using the Illumina HumanExome v1.0, v1.1 and v1.1+custom content SNP arrays.

We called genotypes using Illumina's GenomeStudio software and then zCall⁸, a rare variant caller developed at the Broad Institute, to recover missed rare genotypes. We required that samples have <2% missing genotype calls for common variants (MAF>5%) before applying zCall. Then after applying zCall we removed duplicate SNPs, monomorphic SNPs, SNPs with a low call rate (<98%), and SNPs failing Hardy-Weinberg (p<10⁻⁶). We merged genotype calls from the four shared control cohorts and the AMD cohort by only including SNPs that passed quality control in all 5 cohorts and passed Hardy-Weinberg test (p>10⁻⁶) across all samples. We then used EIGENSTRAT⁴⁷ to check for related samples and generate the first 5 principal components.

### Taqman Genotyping of Selected SNPs

We genotyped Baltimore samples at the Duke University Center for Human Disease Modeling using a custom made TaqMan genotyping assay by Applied Biosystems and with the ABI 7900 Real-Time PCR system.

### Assessing Concordance of Array-Based and Sequencing Based Genotypes

Because rare SNPs are challenging to genotype with array-based platforms, we selected only those alleles with minor allele counts of ≧5 (i.e. allele frequency>0.1%). We also selected only those SNPs with 0% missingness in the array data to ensure the highest possible accuracy for array-based genotypes. To assess concordance we calculated a simple concordance between genotype dosages across individuals with the two different platforms. This correlation-based metric is comparable across allele frequency spectrums.

### Statistical Analyses

Ancestry Informative Markers.

We identified 16,008 ancestry informative markers genotyped on the exome-chip. These SNPs had common allele frequencies (f>5%), and excluded regions in the CFH locus (chr1, 195.5-197.5 MB in HG19 coordinates), the ARMS2 locus (chr10, 123-125 MB), and the Major Histocompatability Complex locus (chr 6, 25-35 MB) loci. We pruned SNPs using the indep option in plink with default parameters (VIF=2, window size=50 SNPs)⁴⁸.

Combining Shared Controls.

For samples genotyped with exome-chip in the France and Boston data sets, we expanded the set of available controls by including shared controls. In order to mitigate the potential effects of population stratification in these replication data sets we included shared controls into each collection by matching on case ancestry. First we applied EIGENSTRAT to these SNP genotypes to calculate principal components to match samples in replication samples together with shared controls⁴⁷. Then we used the first 5 principal components to calculate Euclidean distances between samples in the Boston and France cohorts and shared control samples. Finally, we randomly selected individual case samples in these cohorts and assigned the nearest unassigned shared controls to the selected case's cohort. Shared controls could only be assigned to one of two cohorts. We added one shared control for every two cases to the France collection, and two shared controls for every one case to the Boston replication collection. The resulting expanded Boston and France cohorts had minimal evidence of population stratification (λ_{gc}=1.04 and λ_{gc}=1.06 respectively for ancestry informative markers).

Statistical Framework for Association Testing.

Asymptotic statistics can be inaccurate for rare variants, so we elected to utilize exact statistics instead to test association for individual variants and also for sets of variants in genes (burden tests). We used a strategy similar to Raychaudhuri et al⁵.

For single strata case-control sample collections we used a 2×2 Fisher's exact test to calculate a one-tailed p-value. Assuming a single strata that there are a total of N individuals, of whom n_{case} are cases and nᵥₐᵣᵢₐₙₜ are carriers for the variant, we can calculate the one-tailed significance of observing n_{variant,case} individuals who have the variant and also have advanced AMD as follows:

$${p}_{case \text{-} control} ( {n}_{variant , case} ) = {\sum }_{{n}_{variant , case} \leq ω \leq {n}_{case}} hg ( x , N , {n}_{case} , {n}_{variant} )$$

where hg is the hypergeometric probability distribution assuming that there are nᵥₐᵣᵢₐₙₜ draws from a total of N samples, of which x of a total of n_{case} are drawn.

If multiple case-control strata are present, for example if we are stratifying genotypes of a common variant or combining multiple case-control collections together, we expand the above strategy to calculate an exact p-value. Assume that we observe a total of n_{variant,case} carriers who are affected across all strata then, we can calculate significance as follows:

$${p}_{stratified , case \text{-} control} ( {n}_{variant , case} ) = {\sum }_{sum ( {x}_{1} - {x}_{j} ) \geq {n}_{variant , case}} hg ( {x}_{1} , {N}_{1} , {n}_{1 , case} , {n}_{1 , variant} ) \cdot … \cdot hg ( {x}_{j} , {N}_{j} , {n}_{j , case} , {n}_{j , variant} )$$

Here, for each strata j we have separate numbers of total individuals (Nⱼ), separate numbers of individuals who are cases (n_{j,case}), and heterozygote individuals (n_{j,variant}). So, we calculate the hypergeometric probability for each individual strata for all the possible counts that would result in an equal or greater than n_{variant,case} total number of heterozygotes associated with advanced AMD, and total these probabilities together to obtain a p-value.

For discordant siblings we calculated statistical significance using the binomial distribution. For a given variant, we consider only those pairs of siblings with discordant genotype for the rare variant. The probability under the null that the affected sibling will have the variant is 0.5. We assign each discordant sibling pair a score, sᵢ, which is 1 if the affected sibling has the rare variant or 0 if the unaffected sibling has the rare variant. We obtain an aggregate score by summing over all independent siblings:

$${s}_{siblings} = {\sum }_{i} {s}_{i}$$

Under the null hypothesis, the aggregate score should be distributed according to a binomial distribution. So if there are a total of n_{siblings} we can calculate p_{sibling}, the one-tailed significance:

$${p}_{siblings} ( {s}_{siblings} ) = {\sum }_{x > {s}_{siblings}} binomial ( x , {n}_{siblings} , 0.5 )$$

where the function binomial represents the binomial distribution for x successful draws out of n_{siblings} each with a 0.5 probability.

In order to calculate an aggregate meta-analysis we define a score, S_{aggregate} which is the total of s_{variant,case} across all strata and siblings. We can calculate the probability of obtaining the score s or a more significant score to determine the exact one-tailed p-value:

$${p}_{meta} ( {S}_{aggregate} ) = {\sum }_{{s}_{aggregate} \leq sum ( {x}_{1} - {x}_{j} ) + y} binomial ( y , {n}_{siblings} , 0.5 ) \cdot {\prod }_{j} hg ( {x}_{j} , {N}_{j} , {n}_{j , case} , {n}_{j , variant} )$$

Association Testing.

We filtered variants to include nonsynonymous, splice acceptor-site and donor-site, start lost, stop gained and stop lost; these variants are most likely to alter gene function. Then we identified rare variants (f≦0.01 in either cases or controls). In the single variant association test, we only included rare variants with greater or equal to 5 minor allele counts in our dataset. We used the statistical framework described above. To conduct stratified testing for other nearby common SNPs, we further subdivided strata based on common variant genotypes.

Burden Testing.

To test for increased burden of these annotations, we followed the strategy defined by Li and Leal, using our exact statistical framework¹⁰. We first identified those variants that altered coding sequences, that is, variants that result in missense changes, altered splice acceptor-sites or donor-sites, a start lost, or a stop gained or lost. We selected those variants that were present in with low allele frequencies (f<0.01) in all of our sequenced set of patients. We tested each gene in two ways: (1) assessing if rare variants are increased in cases compared to controls and then (2) assessing if rare variants are increased in controls compared to cases. We used the statistical framework described above to test for aggregate effects.

Defining CFH Haplotypes.

To insure accurate phasing, the individuals in this data set had 0% missing genotype for CFH markers used in our previous study to define haplotypes⁵. For all markers we constructed haplotypes with genotype data from sequencing with Beagle⁴⁹. We selected haplotypes with frequencies>0.5%, and calculated case and control frequencies. For each haplotype we calculated odds ratios and 95% confidence intervals relative to the most frequent haplotype.

Adjusting for Stratification.

We used ancestry informative markers to reassess association statistics for C3 K155Q and rare CFI coding variants and stratification in a subset of sequenced samples with exome-chip data (1,558 cases and 683 controls). We applied EIGENSTRAT To calculate principal components to capture ancestry information and exclude outliers in sequenced samples⁴⁷. For sequenced samples we plotted carriers of C3 K155Q and also of CFI rare variants along the first two components to look for gross evidence of stratification. We also re-assessed two-tailed association statistics by adjusting for the top 5 principal components in a logistic regression framework. Given that logistic regression p-values can be biased for rare events, in the case of C3 K155Q we reported two-tailed significance by calculating beta with the actual data, and comparing it to a null beta distribution defined by permuting case-control labels 1,000,000 times.

### C3 K155Q Functional Assays

Reagents.

For these experiments we obtained purified Factor H(CFH) and Factor I (CFI) (Complement Technologies, Tyler, Tex.); chicken anti-human C3 antibody (Biodesign International, Saco, Me.); goat anti-human C3 antibody (Complement Technologies, Tyler, Tex.); donkey anti-chicken horseradish peroxidase (HRP) linked IgG (Jackson Immunoresearch, Westgrove, Pa.); rabbit anti-goat HRP linked IgG (Sigma, St. Louis, Mo.); murine monoclonal anti-human C3d antibody (Quidel, San Diego, Calif.); and 3,3′,5,5′-Tetramethylbenzidine (TMB) and SuperSignal ELISA substrate (Pierce, Rockford, Ill.).

Protein Expression.

We applied site directed mutagenesis using QuikChange (Agilent Technologies, Santa Clara, Calif.) to modify wildtype C3 cDNA with the 155K allele to instead contain the 155Q mutation. We produced 155K and 155Q C3 proteins by transient transfection of 293T cells with TransIt 293 (Minis, Madison, Wis.) and collected and concentrated cell supernatants three days post-transfection. We treated transfection supernatants with methylamine (MA) to convert native C3 to an inactive, C3b-like form, C3 (MA). We quantified C3 by ELISA, surface plasmon resonance and Western blotting as previously described²⁵.

Ligand Binding Assays.

To assess binding of 155K and 155Q C3 proteins to regulators, we utilized ELISA assays as previously described²⁵. Briefly, we coated either soluble membrane cofactor protein (sMCP), CFH or soluble complement receptor 1 (sCR1) (each at 2 μg/ml) on wells in PBS overnight at 4° C. followed by an incubation with a blocking buffer at 37° C. for 60 minutes. We prepared dilutions of 155K and 155Q C3 proteins in a low salt (25 mM) ELISA buffer. Following incubation at 37° C. for 1 hour, we washed the wells and then applied affinity purified chicken anti-human C3 Ab (1:10,000) (Biodesign International) for 1 hour at 37° C. After washing, we applied HRP linked donkey anti-chicken IgY (1:10,000) for 1 hour at 37° C. Following washing, we added TMB substrate and quantified absorbance at 630 nm.

Surface Plasmon Resonance (SPR) Analysis.

We performed SPR analysis using the BIAcore 2000 (GE Lifesciences, Piscataway, N.J.). Using standard amine coupling technology (GE Lifesciences, Piscataway, N.J.)ₛMCP, CFH and anti-C3d mAb were coupled to individual flow paths. We activated one flow path in each chip without protein as a reference. The running buffer was composed of 10 mM Hepes, pH 7.4, 0.005% Tween-20 and 25 mM NaCl. We injected 155K or the 155Q C3 protein for 90 sec at 30 μl/min and monitored dissociation for 300 sec. We regenerated the chip by injecting 0.5 M NaCl. We analyzed each protein at four concentrations, with a minimum of two injections per concentration. These experiments were performed three times using independently produced and quantitated C3 preparations. We analyzed data using the BIAeval software from BIAcore.

Cofactor Assays.

We assessed cleavage of 155K and 155Q C3 proteins by FI using previously described cofactor assays²⁵. C3 preparations were incubated for 0 to 30 min at 37° C. with CFI (5 ng in MCP and 20 ng in CFH assays) and a cofactor protein MCP (50 ng; recombinant) or CFH (200 ng) in 15 μl of buffer (10 mM Tris, pH 7.4, 150 mM NaCl). To stop the reaction, we added 7 μl of 3× reducing Laemmli sample buffer. The samples were boiled at 95° C. for 5 min, electrophoresed on 10% Tris-glycine polyacrylamide gels, transferred to nitrocellulose and blocked overnight with 5% non-fat dry milk in PBS. We probed the blots with either a 1:10,000 dilution of chicken anti-human C3 Ab followed by HRP-conjugated donkey anti-chicken IgG or a 1:5000 dilution of goat anti-human C3 Ab followed by HRP-conjugated rabbit anti-goat IgG. We developed the blots with SuperSignal substrate.

### REFERENCES

1. Lim, L. S., Mitchell, P., Seddon, J. M., Holz, F. G. & Wong, T. Y. Age-related macular degeneration. Lancet 379, 1728-1738 (2012).
2. Seddon, J. M., Cote, J., Page, W. F., Aggen, S. H. & Neale, M. C. The US twin study of age-related macular degeneration: relative roles of genetic and environmental influences. Arch Ophthalmol 123, 321-327 (2005).
3. Friedman, D. S. et al. Prevalence of age-related macular degeneration in the United States. Arch Ophthalmol 122, 564-572 (2004).
4. Fritsche, L. G. et al. Seven New Loci Associated with Age-Related Macular Degeneration. Nature Genetics In Press,
5. Raychaudhuri, S. et al. A rare penetrant mutation in CFH confers high risk of age-related macular degeneration. Nature Genetics 43, 1232-1236 (2011).
6. Boon, C. J. F. et al. Basal Laminar Drusen Caused by Compound Heterozygous Variants in the CFH Gene. The American Journal of Human Genetics 82, 516-523 (2008).
7. McKenna, A. et al. The Genome Analysis Toolkit: a MapReduce framework for analyzing next-generation DNA sequencing data. Genome Research 20, 1297-1303 (2010).
8. Goldstein, J. I. et al. zCall: a rare variant caller for array-based genotyping: genetics and population analysis. Bioinformatics 28, 2543-2545 (2012).
9. Huyghe, J. R. et al. Exome array analysis identifies new loci and low-frequency variants influencing insulin processing and secretion. Nature Genetics (2012).doi:10.1038/ng.2507
10. Li, B. & Leal, S. M. Methods for Detecting Associations with Rare Variants for Common Diseases: Application to Analysis of Sequence Data. The American Journal of Human Genetics 83, 311-321 (2008).
11. Fagerness, J. A. et al. Variation near complement factor I is associated with risk of advanced AMD. Eur. J. Hum. Genet. 17, 100-104 (2009).
12. Adzhubei, I. A. et al. A method and server for predicting damaging missense mutations. Nature Methods 7, 248-249 (2010).
13. MacArthur, D. G. et al. A systematic survey of loss-of-function variants in human protein-coding genes. Science 335, 823-828 (2012).
14. Bienaime, F. et al. Mutations in components of complement influence the outcome of Factor I-associated atypical hemolytic uremic syndrome. Kidney International 77, 339-349 (2009).
15. Caprioli, J. et al. Genetics of HUS: the impact of MCP, CFH, and IF mutations on clinical presentation, response to treatment, and outcome. Blood 108, 1267-1279 (2006).
16. Nilsson, S. C. et al. A mutation in factor I that is associated with atypical hemolytic uremic syndrome does not affect the function of factor I in complement regulation. Mol. Immunol. 44, 1835-1844 (2007).
17. Geelen, J. et al. A missense mutation in factor I (IF) predisposes to atypical haemolytic uraemic syndrome. Pediatr. Nephrol. 22, 371-375 (2007).
18. Sellier-Leclerc, A.-L. et al. Differential impact of complement mutations on clinical characteristics in atypical hemolytic uremic syndrome. J. Am. Soc. Nephrol. 18, 2392-2400 (2007).
19. Fremeaux-Bacchi, V. et al. Complement factor I: a susceptibility gene for atypical haemolytic uraemic syndrome. J. Med. Genet. 41, e84 (2004).
20. Kavanagh, D. et al. Characterization of mutations in complement factor I (CFI) associated with hemolytic uremic syndrome. Mol. Immunol. 45, 95-105 (2008).
21. Yates, J. R. W. et al. Complement C3 variant and the risk of age-related macular degeneration. N. Engl. J. Med. 357, 553-561 (2007).
22. Maller, J. B. et al. Variation in complement factor 3 is associated with risk of age-related macular degeneration. Nature Genetics 39, 1200-1201 (2007).
23. Nishiguchi, K. M. et al. C9-R95X polymorphism in patients with neovascular age-related macular degeneration. Invest. Ophthalmol. Vis. Sci. 53, 508-512 (2012).
24. Wu, J. et al. Structure of complement fragment C3b-factor H and implications for host protection by complement regulators. Nat. Immunol. 10, 728-733 (2009).
25. Fremeaux-Bacchi, V. et al. Mutations in complement C3 predispose to development of atypical hemolytic uremic syndrome. Blood 112, 4948-4952 (2008).
26. Sánchez-Corral, P. et al. Structural and functional characterization of factor H mutations associated with atypical hemolytic uremic syndrome. Am. J. Hum. Genet. 71, 1285-1295 (2002).
27. Józsi, M. et al. Factor H and atypical hemolytic uremic syndrome: mutations in the C-terminus cause structural changes and defective recognition functions. J. Am. Soc. Nephrol. 17, 170-177 (2006).
28. Manuelian, T. et al. Mutations in factor H reduce binding affinity to C3b and heparin and surface attachment to endothelial cells in hemolytic uremic syndrome. J. Clin. Invest. 111, 1181-1190 (2003).
29. Seddon, J. M., Sharma, S. & Adelman, R. A. Evaluation of the clinical age-related maculopathy staging system. Ophthalmology 113, 260-266 (2006).
30. Seddon, J. M., Cote, J., Davis, N. & Rosner, B. Progression of age-related macular degeneration: association with body mass index, waist circumference, and waist-hip ratio. Arch Ophthalmol 121, 785-792 (2003).
31. Seddon, J. M., Santangelo, S. L., Book, K., Chong, S. & Cote, J. A genomewide scan for age-related macular degeneration provides evidence for linkage to several chromosomal regions. The American Journal of Human Genetics 73, 780-790 (2003).
32. Seddon, J. M. et al. Dietary fat and risk for advanced age-related macular degeneration. Arch Ophthalmol 119, 1191-1199 (2001).
33. Maller, J. et al. Common variation in three genes, including a noncoding variant in CFH, strongly influences risk of age-related macular degeneration. Nature Genetics 38, 1055-1059 (2006).
34. Neale, B. M. et al. Genome-wide association study of advanced age-related macular degeneration identifies a role of the hepatic lipase gene (LIPC). Proc. Natl. Acad. Sci. U.S.A. 107, 7395-7400 (2010).
35. Yu, Y. et al. Common variants near FRK/COL10A1 and VEGFA are associated with advanced age-related macular degeneration. Hum. Mol. Genet. 20, 3699-3709 (2011).
36. Yang, Z. et al. Genetic and functional dissection of HTRA1 and LOC387715 in age-related macular degeneration. PLoS Genet. 6, e1000836 (2010).
37. Yang, Z. et al. Toll-like receptor 3 and geographic atrophy in age-related macular degeneration. N. Engl. J. Med. 359, 1456-1463 (2008).
38. Chen, W. et al. Genetic variants near TIMP3 and high-density lipoprotein-associated loci influence susceptibility to age-related macular degeneration. Proc. Natl. Acad. Sci. U.S.A. 107, 7401-7406 (2010).
39. 1000 Genomes Project Consortium A map of human genome variation from population-scale sequencing. Nature 467, 1061-1073 (2010).
40. Daly, A. K. et al. HLA-B*5701 genotype is a major determinant of drug-induced liver injury due to flucloxacillin. Nature Genetics 41, 816-819 (2009).
41. Sklar, P. et al. Whole-genome association study of bipolar disorder. Mol. Psychiatry. 13, 558-569 (2008).
42. Rivas, M. A. et al. Deep resequencing of GWAS loci identifies independent rare variants associated with inflammatory bowel disease. Nature Genetics 43, 1066-1073 (2011).
43. Li, H. & Durbin, R. Fast and accurate short read alignment with Burrows-Wheeler transform. Bioinformatics 25, 1754-1760 (2009).
44. DePristo, M. A. et al. A framework for variation discovery and genotyping using next-generation DNA sequencing data. Nature Genetics 43, 491-498 (2011).
45. Tennessen, J. A. et al. Evolution and Functional Impact of Rare Coding Variation from Deep Sequencing of Human Exomes. Science 337, 64-69 (2012).
46. Cingolani, P. et al. A program for annotating and predicting the effects of single nucleotide polymorphisms, SnpEff: SNPs in the genome of Drosophila melanogaster strain w1118; iso-2; iso-3. Fly (Austin) 6, 80-92 (2012).
47. Price, A. L. et al. Principal components analysis corrects for stratification in genome-wide association studies. Nature Genetics 38, 904-909 (2006).
48. Purcell, S. et al. PLINK: a tool set for whole-genome association and population-based linkage analyses. Am. J. Hum. Genet. 81, 559-575 (2007).
49. Browning, S. R. Multilocus association mapping using variable-length Markov chains. Am. J. Hum. Genet. 78, 903-913 (2006).

SUPPLEMENTARY TABLE 1
Association of known AMD alleles in sequencing data.

| | Published SNP | | Coordinates | | | | Reported | | |
|---|---|---|---|---|---|---|---|---|---|
| gene | association | Proxy SNP | (HG19) | r² | RAF_A | RAF_U | OR | OR | P |
| CFH | rs10737680 | rs10737680 | 196679455 | 1.00 | 0.80 | 0.58 | 2.43 | 3.02 | 9.8E−62 |
| ADAMTS9 | rs6795735 | rs11710526 | 64728998 | 0.18 | 0.88 | 0.88 | 1.10 | 1.02 | 0.84 |
| COL8A1, | rs13081855 | rs9883702 | 99397141 | 1.00 | 0.14 | 0.12 | 1.23 | 1.23 | 0.031 |
| FILIP1L | | | | | | | | | |
| CFI | rs4698775 | rs13846 | 110605744 | 0.90 | 0.32 | 0.28 | 1.14 | 1.20 | 0.0085 |
| FRK, COL10A1 | rs3812111 | rs1064583 | 116446576 | 1.00 | 0.63 | 0.56 | 1.10 | 1.34 | 4.5E−06 |
| IER3, DDR1 | rs3130783 | rs3094108 | 30850395 | 0.31 | 0.20 | 0.20 | 1.16 | 1.01 | 0.92 |
| CFB, C2 | rs429608 | rs541862 | 31916951 | 0.53 | 0.96 | 0.91 | 1.74 | 2.17 | 1.2E−10 |
| VEGFA | rs943080 | rs62401162 | 43746949 | 0.02 | 0.83 | 0.82 | 1.15 | 1.09 | 0.31 |
| TNFRSF10A | rs13278062 | rs4872077 | 23058188 | 0.27 | 0.32 | 0.30 | 1.15 | 1.10 | 0.16 |
| TGFB1 | rs334353 | rs334348 | 101912471 | 1.00 | 0.76 | 0.75 | 1.13 | 1.05 | 0.50 |
| ARMS2 | rs10490924 | rs10490924 | 124214448 | 1.00 | 0.45 | 0.21 | 2.76 | 3.13 | 1.9E−58 |
| B3GALTL | rs9542236 | rs9542307 | 31821256 | 0.79 | 0.49 | 0.46 | 1.10 | 1.10 | 0.14 |
| RAD51B | rs8017304 | rs2074563 | 68935112 | 0.05 | 0.38 | 0.38 | 1.11 | 1.01 | 0.92 |
| LIPC | rs920915 | rs17821310 | 58702864 | 0.15 | 0.81 | 0.77 | 1.13 | 1.28 | 0.0012 |
| CETP | rs1864163 | rs7205804 | 57004889 | 0.26 | 0.48 | 0.43 | 1.22 | 1.21 | 0.0021 |
| APOE | rs4420638 | rs769449 | 45410002 | 0.53 | 0.91 | 0.89 | 1.30 | 1.36 | 0.0025 |
| C3 | rs2230199 | rs2230199 | 6718387 | 1.00 | 0.29 | 0.20 | 1.42 | 1.61 | 1.9E−10 |
| TIMP3 | rs5749482 | rs1427384 | 33257322 | 0.04 | 0.28 | 0.27 | 1.31 | 1.04 | 0.62 |
| SLC16A8 | rs8135665 | rs67460670 | 38480007 | 0.86 | 0.20 | 0.19 | 1.15 | 1.05 | 0.57 |

SUPPLEMENTARY TABLE 2
CFI alleles. We list each of the alleles that we identified in CFI that alter coding variants. For each variant we list the HG19 coordinates,
the rsIDs if available, the reference and alternate alleles, the amino acid change conferred, and the codon change conferred. We also
list the frequency in the Exome Variant Server (http://evs.gs.washington.edu/EVS/), whether the variant has been seen in atypical
hemolytic uremic syndrome, the number of AMD cases and controls with the rare variant, and the PolyPhen annotation.

| Coordinates | | | | Amino Acid | Codon | | Reported | Case | Control | PolyPhen |
|---|---|---|---|---|---|---|---|---|---|---|
| (HG19) | rsID | REF | ALT | Change | Change | ESP-CEU | HUS | (n = 1,712) | (n = 781) | Annotation |
| chr4: 110662063 | | G | A | Q580* | Cag/Tag | | | 1 | 0 | Loss of Function |
| chr4: 110662068 | | A | G | I578T | aTt/aCt | | | 1 | 0 | Probably Damaging |
| chr4: 110662140 | | T | A | E554V | gAg/gTg | | | 3 | 0 | Probably Damaging |
| chr4: 110662144 | rs113460688 | G | A | P553S | Cca/Tca | A = 15/ | YES | 18 | 2 | Benign |
| | | | | | | G = 8585 | | | | |
| chr4: 110662173 | | A | G | V543A | gTt/gCt | | | 1 | 0 | Possibly Damaging |
| chr4: 110662178 | | C | T | W541* | tgG/tgA | | | 1 | 0 | Loss of Function |
| chr4: 110662179 | | C | T | W541* | tGg/tAg | | | 1 | 0 | Loss of Function |
| chr4: 110663647 | | C | T | G512S | Ggt/Agt | | | 1 | 0 | Probably Damaging |
| chr4: 110663677 | | G | A | R502C | Cgt/Tgt | | | 1 | 0 | Probably Damaging |
| chr4: 110663707 | | T | G | I492L | Ata/Cta | G = 1/ | | 1 | 0 | Benign |
| | | | | | | T = 8595 | | | | |
| chr4: 110663722 | | C | A | G487C | Ggt/Tgt | | | 1 | 0 | Probably Damaging |
| chr4: 110667377 | | C | G | Splice | | G = 1/ | | 3 | 0 | Loss of Function |
| | | | | Site Donor | | C = 8599 | | | | |
| chr4: 110667378 | | C | G | D477H | Gat/Cat | | | 2 | 0 | Possibly Damaging |
| chr4: 110667387 | rs121964913 | G | A | R474* | Cga/Tga | A = 1/ | YES | 1 | 0 | Loss of Function |
| | | | | | | G = 8599 | | | | |
| chr4: 110667408 | | A | G | C467R | Tgc/Cgc | | | 1 | 0 | Probably Damaging |
| chr4: 110667431 | | T | G | Y459S | tAc/tCc | | YES | 1 | 0 | Possibly Damaging |
| chr4: 110667485 | rs41278047 | T | C | K441R | aAa/aGa | C = 45/ | | 23 | 3 | Benign |
| | | | | | | T = 8555 | | | | |
| chr4: 110667554 | rs121964912 | T | A | H418L | cAt/cTt | A = 1/ | | 2 | 0 | Probably Damaging |
| | | | | | | T = 8599 | | | | |
| chr4: 110667561 | rs61733901 | T | G | I416L | Att/Ctt | G = 1/ | YES | 1 | 0 | Benign |
| | | | | | | T = 8599 | | | | |
| chr4: 110667590 | rs74817407 | C | T | R406H | cGt/cAt | T = 8/ | | 1 | 0 | Possibly Damaging |
| | | | | | | C = 8592 | | | | |
| chr4: 110667600 | rs139881195 | C | T | D403N | Gac/Aac | T = 1/ | YES | 1 | 0 | Benign |
| | | | | | | C = 8599 | | | | |
| chr4: 110667612 | | A | G | W399R | Tgg/Cgg | | | 2 | 0 | Benign |
| chr4: 110667641 | | C | T | R389H | cGt/cAt | | | 1 | 0 | Benign |
| chr4: 110670400 | | C | A | W374C | tgG/tgT | | | 1 | 0 | Probably Damaging |
| chr4: 110670416 | | T | G | Y369S | tAt/tCt | | YES | 1 | 0 | Probably Damaging |
| chr4: 110670437 | | C | G | G362A | gGa/gCa | | | 0 | 1 | Benign |
| chr4: 110670459 | | C | T | V355M | Gtg/Atg | | | 0 | 1 | Probably Damaging |
| chr4: 110670680 | | A | G | I340T | aTt/aCt | | YES | 1 | 0 | Probably Damaging |
| chr4: 110670749 | | C | T | R317Q | cGg/cAg | | | 1 | 0 | Benign |
| chr4: 110670750 | rs121964917 | G | A | R317W | Cgg/Tgg | A = 2/ | YES | 2 | 1 | Benign |
| | | | | | | G = 8598 | | | | |
| chr4: 110673634 | | G | T | D310E | gaC/gaA | | | 1 | 0 | Possibly Damaging |
| chr4: 110678925 | rs11098044 | T | C | T300A | Act/Gct | C = 8486/ | | 4 | 2 | Benign |
| | | | | | | T = 8 | | | | |
| chr4: 110681450 | | C | T | G287R | Ggg/Agg | T = 1/ | | 3 | 0 | Probably Damaging |
| | | | | | | C = 8599 | | | | |
| chr4: 110681470 | | C | T | G280D | gGt/gAt | | | 1 | 0 | Possibly Damaging |
| chr4: 110681521 | | C | A | G263V | gGc/gTc | | | 1 | 0 | Probably Damaging |
| chr4: 110681527 | rs112534524 | C | T | G261D | gGc/gAc | T = 17/ | YES | 9 | 3 | Benign |
| | | | | | | C = 8583 | | | | |
| chr4: 110681679 | | C | T | A258T | Gca/Aca | T = 4/ | | 3 | 0 | Possibly Damaging |
| | | | | | | C = 8596 | | | | |
| chr4: 110681708 | | C | T | G248E | gGa/gAa | | | 1 | 0 | Probably Damaging |
| chr4: 110681732 | rs146444258 | G | C | A240G | gCc/gGc | C = 5/ | YES | 12 | 1 | Probably Damaging |
| | | | | | | G = 8595 | | | | |
| chr4: 110681763 | | C | T | V230M | Gtg/Atg | | | 1 | 0 | Probably Damaging |
| chr4: 110681766 | | A | G | C229R | Tgt/Cgt | | | 1 | 0 | Probably Damaging |
| chr4: 110681781 | | C | T | D224N | Gat/Aat | | | 1 | 0 | Benign |
| chr4: 110681789 | | G | T | S221Y | tCt/tAt | T = 1/ | | 1 | 0 | Possibly Damaging |
| | | | | | | G = 8599 | | | | |
| chr4: 110682680 | | C | G | Q217H | caG/caC | | | 0 | 1 | Possibly Damaging |
| chr4: 110682715 | | A | T | Y206N | Tac/Aac | T = 1/ | | 1 | 0 | Benign |
| | | | | | | A = 8599 | | | | |
| chr4: 110682723 | rs138346388 | G | A | T203I | aCt/aTt | A = 7/ | | 2 | 0 | Benign |
| | | | | | | G = 8593 | | | | |
| chr4: 110682726 | rs149215929 | C | A | R202I | aGa/aTa | T = 0/ | | 0 | 1 | Possibly Damaging |
| | | | | | | C = 8600 | | | | |
| chr4: 110682739 | | A | G | F198L | Ttt/Ctt | | | 1 | 0 | Benign |
| chr4: 110682771 | rs143366614 | C | T | R187Q | cGa/cAa | T = 3/ | | 1 | 0 | Possibly Damaging |
| | | | | | | C = 8597 | | | | |
| chr4: 110682781 | | C | T | V184M | Gtg/Atg | | | 0 | 1 | Possibly Damaging |
| chr4: 110682801 | | T | A | N177I | aAt/aTt | | | 1 | 0 | Benign |
| chr4: 110685721 | | C | T | V152M | Gtg/Atg | T = 1/ | | 2 | 0 | Probably Damaging |
| | | | | | | C = 8599 | | | | |
| chr4: 110685795 | | A | G | V127A | gTt/gCt | | | 1 | 0 | Possibly Damaging |
| chr4: 110685820 | rs141853578 | C | T | G119R | Gga/Aga | T = 11/ | YES | 7 | 1 | Probably Damaging |
| | | | | | | C = 8589 | | | | |
| chr4: 110687712 | | T | G | E109A | gAa/gCa | | | 1 | 0 | Benign |
| chr4: 110687847 | | G | A | P64L | cCg/cTg | | | 1 | 0 | Probably Damaging |
| chr4: 110687890 | rs144082872 | G | C | P50A | Cca/Gca | C = 1/ | YES | 2 | 0 | Probably Damaging |
| | | | | | | G = 8599 | | | | |
| chr4: 110687908 | | C | T | D44N | Gat/Aat | T = 1/ | | 1 | 0 | Benign |
| | | | | | | C = 8599 | | | | |
| chr4: 110687919 | | T | C | H40R | cAc/cGc | | | 1 | 0 | Possibly Damaging |

SUPPLEMENTARY TABLE 3
Associated variants in discovery sequencing of Boston samples. Here we list the individual variants that are associated with AMD risk or protection.
For each variant we list the HG19 coordinates, the rsID (if available), the reference and alternate alleles, the gene that the variant is
in, and the amino acid change. Then we list the allele counts of the reference allele (R) and the alternate allele (A) in discovery for disease
(AMD) and controls (cont). We separate allele counts down by case-control samples and also discordant sibling pairs. We also show the one-
tailed p-value in the discovery and three replication cohorts, and the exact replication p-value and the joint analysis p-value.

| | | | | | | | Sequencing Discovery | | | | | |
|---|---|---|---|---|---|---|---|---|---|---|---|---|
| | | | | | | | Case-Control | | | | Sibling Pairs | |
| | Coordinates | | | | | Amino Acid | (allele counts) | | | | (allele counts) | |
| | (HG19) | rsID | REF | ALT | GENE | Chance | AMD-R | Cont-R | AMD-A | Cont-A | AMD-R | Cont-R |
| Risk | chr19: 6718146 | rs147859257 | T | G | C3 | K155Q | 3312 | 1489 | 40 | 1 | 71 | 71 |
| | chr5: 39331894 | rs34882957 | G | A | C9 | P167S | 3299 | 1479 | 53 | 11 | 71 | 72 |
| | chr1: 196716375 | rs121913059 | C | T | CFH | R1210C | 3336 | 1489 | 16 | 1 | 71 | 72 |
| | chr6: 31939416 | rs11558689 | T | C | DOM3Z | T13A | 3335 | 1490 | 17 | 0 | 72 | 71 |
| | chr3: 49548225 | rs145403829 | G | C | DAG1 | L86F | 3330 | 1488 | 22 | 2 | 72 | 72 |
| Protection | chr1: 196928188 | rs41310132 | A | G | CFHR2 | Y264C | 3333 | 1459 | 19 | 31 | 72 | 71 |
| | chr10: 1284303 | rs146452150 | C | T | ADARB2 | G418S | 3341 | 1473 | 11 | 17 | 72 | 71 |
| | chr1: 196712596 | rs35274867 | A | T | CFH | N1050Y | 3330 | 1463 | 22 | 27 | 72 | 71 |
| | chr6: 32065113 | rs61746206 | C | T | TNXB | A173T | 3331 | 1467 | 21 | 23 | 72 | 72 |
| | chr14: 105179788 | | A | C | INF2 | K962T | 3352 | 1485 | 0 | 5 | 72 | 72 |
| | chr8: 19818551 | rs5934 | G | A | LPL | A413T | 3351 | 1484 | 1 | 6 | 72 | 72 |
| | chr19: 6736607 | rs4807897 | A | G | GPR108 | L79P | 3325 | 1465 | 27 | 25 | 72 | 71 |
| | chr6: 32041621 | rs6910390 | G | A | TNXB | T1495I | 3347 | 1480 | 5 | 10 | 72 | 72 |
| | chr1: 151503071 | rs138238589 | G | A | CGN | R807Q | 3339 | 1474 | 13 | 16 | 72 | 72 |
| | chr6: 31802938 | | C | T | C6orf48 | S71L | 3335 | 1472 | 17 | 18 | 72 | 71 |
| | chr1: 94528774 | rs145525174 | C | T | ABCA4 | V552I | 3348 | 1482 | 4 | 8 | 72 | 71 |
| | chr13: 111117923 | | C | T | COL4A2 | P650S | 3345 | 1479 | 7 | 11 | 72 | 72 |
| | chr17: 78896587 | rs144632265 | G | A | RPTOR | A704T | 3340 | 1476 | 12 | 14 | 72 | 71 |
| | chr6: 31727989 | rs61748589 | A | G | MSH5 | K135R | 3332 | 1469 | 20 | 21 | 70 | 71 |
| | chr9: 125152621 | rs5794 | G | A | PTGS1 | V372I | 3338 | 1474 | 14 | 16 | 71 | 71 |
| | chr1: 196965193 | rs139017763 | G | A | CFHR5 | G278S | 3342 | 1477 | 10 | 13 | 72 | 72 |

| | | | Sequencing Discovery | | One tailed exact p-value | | | | | |
|---|---|---|---|---|---|---|---|---|---|---|
| | | | Sibling Pairs | | | | | | | |
| | Coordinates | | (allele counts) | | Discovery | | Replication | | Joint | |
| | (HG19) | rsID | AMD-A | Cont-A | Boston | Boston | France | JHU | All | |
| Risk | chr19: 6718146 | rs147859257 | 1 | 1 | 4.8E−6 | 9.3E−4 | 0.018 | 0.24 | 3.5E−5 | 5.2E−9 |
| | chr5: 39331894 | rs34882957 | 1 | 0 | 6.8E−3 | 2.3E−3 | 4.5E−3 | 0.22 | 2.4E−5 | 6.5E−7 |
| | chr1: 196716375 | rs121913059 | 1 | 0 | 9.0E−3 | — | — | 0.06 | 0.061 | 7.4E−4 |
| | chr6: 31939416 | rs11558689 | 0 | 1 | 9.0E−3 | 0.13 | 0.14 | — | 0.038 | 1.2E−3 |
| | chr3: 49548225 | rs145403829 | 0 | 0 | 9.5E−3 | 0.41 | 0.99 | — | 0.87 | 0.31 |
| Protection | chr1: 196928188 | rs41310132 | 0 | 1 | 2.5E−6 | — | — | 0.15 | 0.15 | 1.2E−6 |
| | chr10: 1284303 | rs146452150 | 0 | 1 | 5.9E−4 | 0.77 | 0.44 | — | 0.66 | 0.04 |
| | chr1: 196712596 | rs35274867 | 0 | 1 | 6.9E−4 | 1.2E−4 | 0.027 | — | 2.2E−5 | 7.3E−8 |
| | chr6: 32065113 | rs61746206 | 0 | 0 | 2.2E−3 | — | — | — | — | — |
| | chr14: 105179788 | | 0 | 0 | 2.7E−3 | — | — | — | — | — |
| | chr8: 19818551 | rs5934 | 0 | 0 | 4.3E−3 | 0.98 | 1.00 | — | 0.98 | 0.14 |
| | chr19: 6736607 | rs4807897 | 0 | 1 | 4.4E−3 | 0.029 | 0.80 | — | 0.15 | 6.5E−3 |
| | chr6: 32041621 | rs6910390 | 0 | 0 | 4.4E−3 | — | — | — | — | — |
| | chr1: 151503071 | rs138238589 | 0 | 0 | 5.2E−3 | 0.14 | 0.75 | — | 0.39 | 0.046 |
| | chr6: 31802938 | | 0 | 1 | 5.6E−3 | — | — | — | — | — |
| | chr1: 94528774 | rs145525174 | 0 | 1 | 6.5E−3 | 0.71 | 0.49 | — | 0.59 | 0.055 |
| | chr13: 111117923 | | 0 | 0 | 7.3E−3 | — | — | — | — | — |
| | chr17: 78896587 | rs144632265 | 0 | 1 | 7.7E−3 | — | — | — | — | — |
| | chr6: 31727989 | rs61748589 | 2 | 1 | 8.0E−3 | 0.50 | 0.39 | — | 0.38 | 0.03 |
| | chr9: 125152621 | rs5794 | 1 | 1 | 8.0E−3 | 0.77 | 0.55 | — | 0.68 | 0.18 |
| | chr1: 196965193 | rs139017763 | 0 | 0 | 8.9E−3 | — | — | 1.1E−3 | 1.1E−3 | 1.0E−4 |

SUPPLEMENTARY TABLE 4
Replication samples. For each sample collection we list the total
number of cases, examined controls, and shared controls.

| | | | | | | Discordant | Total | Total |
|---|---|---|---|---|---|---|---|---|
| | | Genotyping | Cases | Examined | Shared | Sibling pairs | Cases | Controls |
| Discovery | Boston | Sequencing | 1,676 | 745 | | 36 | | |
| Replication | Boston | Exome Chip | 746 | 542 | 1491 | 13 | 759 | 2,046 |
| | France | Exome Chip | 953 | 204 | 476 | — | 953 | 680 |
| | Baltimore | TaqMan | 515 | 162 | | — | 515 | 162 |

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

What is claimed is:

1. A method for determining a risk of age-related macular degeneration (AMD) in a human patient, the method comprising detecting in a sample from the human patient the presence of polymorphism rs147859257 (SEQ ID NO: 17), and correlating the presence of polymorphism rs147859257 (SEQ ID NO: 17) to an increased risk of AMD in the human patient.

2. A method for determining a risk of age-related macular degeneration (AMD) in a human patient, the method comprising detecting in a sample from the human patient the presence of polymorphism rs34882957 (SEQ ID NO: 18), and correlating the presence of polymorphism rs34882957 (SEQ ID NO: 18) to an increased risk of AMD in the human patient.

3. A method for determining a risk of age-related macular degeneration (AMD) in a human patient, the method comprising detecting in a sample from the human patient the presence of a polymorphism listed in Supplementary Table 2 (SEQ ID NOS 1-16, respectively, in order of appearance), and correlating the presence of the polymorphism to AMD risk in the human patient.

4. The method of claim 1, wherein the detecting is performed using a hybridization assay.

5. The method of claim 1, wherein the detecting is performed using a microarray.

6. The method of claim 1, wherein the detecting is performed using an antibody.

7. The method of claim 1, wherein the detecting is performed by sequencing.

8. The method of claim 1, wherein the presence of a variant allele is indicative that the patient will progress to advanced AMD.

9. The method of claim 1, wherein the presence of a variant allele is indicative that the patient will progress to advanced AMD.

10. The method of claim 1, wherein the sample is from a subject who is determined to be at risk for developing age-related macular degeneration due to one or more patient specific risk factors.

11. The method of claim 10, wherein the one or more patient specific risk factors is genetic, environmental, demographic, or behavioral.

12. The method of claim 11, wherein the one or more environmental risk factors are selected from the group consisting of: obesity, smoking, vitamin and dietary supplement intake, use of alcohol or drugs, poor diet, a sedentary lifestyle, and combinations thereof.

13. The method of claim 11, wherein the genetic risk factor is selected from the group consisting of: family history of AMD, presentation of AMD symptoms, detection of one or more AMD risk alleles in the patient, and combinations thereof.

14. The method of claim 11, wherein the demographic factor is selected from the group consisting of: age, sex, education level, income level, marital status, occupation, religion, birth rate, death rate, average size of a family, average age at marriage.
